(19) United States
(12) Patent Application Publication
(10) Pub. No.: US 2002/0013295 A1
(43) Pub. Date: Jan. 31, 2002

(54) Naaladase inhibitors for treating amyotrophic lateral sclerosis

(76) Inventors: Barbara S. Slusher, Kingsville, MD (US); Krystyna Wozniak, Bel Air, MD (US)

Correspondence Address: LYON & LYON LLP, 633 WEST FIFTH STREET, SUITE 4700, LOS ANGELES, CA, 90071, US

(21) Appl. No.: 09/866,729

(22) Filed: May 30, 2001

Related U.S. Application Data
(60) Provisional application No. 60/207,319, filed on May 30, 2000.

(51) Int. Cl. A61K 31/675; A61K 31/66; A61K 31/415; A61K 31/197

(52) U.S. Cl. 514/79; 514/94; 514/114; 514/406; 514/561

(57) ABSTRACT

The present invention relates to pharmaceutical compositions and methods for treating amyotrophic lateral sclerosis using NAALADase inhibitors.

This application claims the benefit of U.S. Provisional Application No. 60/207,317 filed on May 30, 2000.

The present invention relates to pharmaceutical compositions and methods for treating amyotrophic lateral sclerosis (“ALS” ) using NAALADase inhibitors.

The NAALADase enzyme, also known as prostate specific membrane antigen (“PSM” or “PSMA” ) and human glutamate carboxypeptidase II (“GCP II” ), catalyzes the hydrolysis of the neuropeptide N-acetyl-aspartyl-glutamate (“NAAG”) to N-acetyl-aspartate (“NAA”) and glutamate. Based upon amino acid sequence homology, NAALADase has been assigned to the M28 family of peptidases.

NAAG and NAALADase have been implicated in the pathogenesis of ALS and in the pathologically similar animal disease called Hereditary Canine Spinal Muscular Atrophy (“HCSMA” ). Studies show that concentrations of NAAG and its metabolites (NAA, glutamate) are elevated two- to three-fold in cerebral spinal fluid from ALS patients and HCSMA dogs.

The etiology of ALS has been linked to alterations of glutamatergic neurotransmission. Post mortem studies on ALS patients show elevated measurements of glutamate in serum, cerebrospinal fluid and brain; decreased high-affinity glutamate uptake by synaptosomes from spinal cord and motor cortex; and decreased expression of the primarily glial GLT-1 glutamate transporter. The therapeutic benefit of putative glutamate inhibitors, riluzole and gabapentin, on the survival of mutant SOD1 transgenic mice also implicates glutamate in the pathogenesis of ALS.

### SUMMARY OF THE INVENTION

The present invention relates to a method for treating amyotrophic lateral sclerosis (“ALS”) comprising administering an effective amount of a NAALADase inhibitor to a mammal in need of such treatment.

The present invention further relates to a pharmaceutical composition comprising:

(i) an effective amount of a NAALADase inhibitor for treating amyotrophic lateral sclerosis (ALS); and

(ii) a pharmaceutically acceptable carrier.

### BRIEF DESCRIPTION OF THE DRAWINGS

FIG. 1 is a bar graph plotting the percent of transgenic mice at 210 days of age that exhibited limb shaking after treatment with 2-(3-sulfanylpropyl)pentanedioic acid (“Compound B” ) or a vehicle.

FIG. 2 is a bar graph plotting the gait, measured on an arbitrary scale ranging from 0 to 3, of transgenic mice at 210 days of age after treatment with Compound B or a vehicle.

FIG. 3 is a bar graph plotting hind limbs dragging, measured on an arbitrary scale ranging from 0 to 3, of transgenic mice at 210 days of age after treatment with Compound B or a vehicle.

FIG. 4 is a bar graph plotting the crossing of hind limbs, measured on an arbitrary scale ranging from 0 to 3, of transgenic mice at 210 days of age after treatment with Compound B or a vehicle.

FIG. 5 is a bar graph plotting the righting reflex of transgenic mice, measured by the time (seconds) it took the mice to right themselves when placed on their sides, at 210 days of age after treatment with Compound B or a vehicle.

FIG. 6 is a graph plotting the percent of transgenic mice treated with Compound B or a vehicle that died against the age of the mice (days).

FIG. 7 is a Kaplan-Meier survival graph plotting the percent of transgenic mice treated with Compound B or a vehicle that survived against the number of days that the mice were on study therapy.

### DETAILED DESCRIPTION OF THE INVENTION

### Definitions

“Alkyl” refers to a branched or unbranched saturated hydrocarbon chain comprising a designated number of carbon atoms. For example, C₁-C₉ alkyl is a straight or branched hydrocarbon chain containing 1 to 9 carbon atoms, and includes but is not limited to substituents such as methyl, ethyl, propyl, iso-propyl, butyl, iso-butyl, tert-butyl, n-pentyl, n-hexyl, and the like, unless otherwise indicated.

“Alkenyl” refers to a branched or unbranched unsaturated hydrocarbon chain comprising a designated number of carbon atoms. For example, C₂-C₉ alkenyl is a straight or branched hydrocarbon chain containing 2 to 9 carbon atoms having at least one double bond, and includes but is not limited to substituents such as ethenyl, propenyl, iso-propenyl, butenyl, iso-butenyl, tert-butenyl, n-pentenyl, n-hexenyl, and the like, unless otherwise indicated.

“Alkoxy” refers to the group —OR wherein R is alkyl as herein defined. Preferably, R is a branched or unbranched saturated hydrocarbon chain containing 1 to 9 carbon atoms.

“Carbocycle” refers to a hydrocarbon, cyclic moiety having one or more closed ring(s) that is/are alicyclic, aromatic, fused and/or bridged. Examples include cyclopropane, cyclobutane, cyclopentane, cyclohexane, cycloheptane, cyclopentene, cyclohexene, cycloheptene, cycloctene, benzyl, naphthene, anthracene, phenanthracene, biphenyl and pyrene.

“Aryl” refers to an aromatic, hydrocarbon cyclic moiety having one or more closed ring(s). Examples include, without limitation, phenyl, naphthyl, anthracenyl, phenanthracenyl, biphenyl and pyrenyl.

“Heterocycle” refers to a cyclic moiety having one or more closed ring(s) that is/are alicyclic, aromatic, fused and/or bridged, with one or more heteroatom(s) (for example, sulfur, nitrogen or oxygen) in at least one of the rings. Examples include, without limitation, pyrrolidine, pyrrole, thiazole, thiophene, piperidine, pyridine, isoxazolidine and isoxazole.

“Heteroaryl” refers to an aromatic, cyclic moiety having one or more closed ring(s) with one or more heteroatom(s) (for example, sulfur, nitrogen or oxygen) in at least one of the rings. Examples include, without limitation, pyrrole, thiophene, pyridine and isoxazole.

“Linking group” refers to a moiety that connects the terminal group with the benzene ring in the compounds of formula VI, without compromising with the pharmacological or biological activity of the overall compound.

“Metal binding group” refers to a functional group capable of interacting with metal ion(s), such as Co²⁺, Ni²⁺, Mn²⁺, Cu²⁺, Zn²⁺, Mg²⁺, Fe²⁺, Fe³⁺, or Al^{3+.} Metal binding groups include without limitation amines (e.g. ethylenediamine), aldehydes, ketones, carboxylic acids (e.g. ethylenediaminetetraacetic acid (“EDTA” )), thiols, phosphorus derivatives and hydroxamic acids.

“Derivative” refers to a substance produced from another substance either directly or by modification or partial substitution.

“Effective amount” refers to the amount required to produce the desired effect.

“Therapeutically effective amount” refers to the amount required to treat ALS in an animal or a mammal.

“Halo” refers to at least one fluoro, chloro, bromo or iodo moiety.

“Isosteres” refer to elements, functional groups, substitutents, molecules or ions having different molecular formulae but exhibiting similar or identical physical properties. For example, tetrazole is an isostere of carboxylic acid because it mimics the properties of carboxylic acid even though they both have different molecular formulae. Typically, two isosteric molecules have similar or identical volumes and shapes. Ideally, isosteric compounds should be isomorphic and able to co-crystallize. Other physical properties that isosteric compounds usually share include boiling point, density, viscosity and thermal conductivity. However, certain properties are usually different: dipolar moments, polarity, polarization, size and shape since the external orbitals may be hybridized differently. The term “isosteres” encompass “bioisosteres”.

“Bioisosteres” are isosteres that, in addition to their physical similarities, share some common biological properties. Typically, bioisosteres interact with the same recognition site or produce broadly similar biological effects.

“Carboxylic acid isosteres” include without limitation direct derivatives such as hydroxamic acids, acyl-cyanamides and acylsulfonamides; planar acidic heterocycles such as tetrazoles, mercaptoazoles, sulfinylazoles, sulfonylazoles, isoxazoles, isothiazoles, hydroxythiadiazoles and hydroxychromes; and nonplanar sulfur- or phosphorus-derived acidic functions such as phosphinates, phosphonates, phosphonamides, sulphonates, sulphonamides, and acylsulphonamides.

“Metabolite” refers to an intermediate or product resulting from metabolism.

“NAAG” refers to N-acetyl-aspartyl-glutamate, an important peptide component of the brain, with levels comparable to the major inhibitor neurotransmitter gamma-aminobutyric acid (“GABA” ). NAAG is neuron-specific, present in synaptic vesicles and released upon neuronal stimulation in several systems presumed to be glutamatergic. Studies suggest that NAAG may function as a neurotransmitter and/or neuromodulator in the central nervous system, or as a precursor of the neurotransmitter glutamate. In addition, NAAG is an agonist at group II metabotropic glutamate receptors, specifically mGluR3 receptors; when attached to a moiety capable of inhibiting NAALADase, it is expected that metabotropic glutamate receptor ligands will provide potent and specific NAALADase inhibitors.

“NAALADase” refers to N-acetylated α-linked acidic dipeptidase, a membrane bound metallopeptidase that catabolizes NAAG to N-acetylaspartate (“NAA”) and glutamate (“GLU”):

Catabolism of NAAG by NAALADase

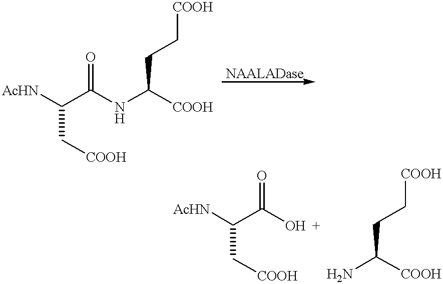

NAALADase has been assigned to the M28 peptidase family and is also called prostate specific membrane antigen (“PSM” ) or human glutamate carboxypeptidase II (“GCP II” ), EC number 3.4.17.21. It is believed that NAALADase is a co-catalytic zinc/zinc metallopeptidase. NAALADase shows a high affinity for NAAG with a Km of 540 nM. If NAAG is a bioactive peptide, then NAALADase may serve to inactivate NAAG'S synaptic action. Alternatively, if NAAG functions as a precursor for glutamate, the primary function of NAALADase may be to regulate synaptic glutamate availability.

“Pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, preferably non-toxic, that would be suitable for use in a pharmaceutical composition.

“Pharmaceutically acceptable equivalent” includes, without limitation, pharmaceutically acceptable salts, hydrates, metabolites, prodrugs, and isosteres. Many pharmaceutically acceptable equivalents are expected to have the same or similar in vitro or in vivo activity as the inventive compounds.

“Pharmaceutically acceptable salt” refers to a salt of the inventive compounds that possesses the desired pharmacological activity and that is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. The basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using methods known in the art, such as those described by Burger's Medicinal Chemistry and Drug Chemistry, Fifth Ed., Vol. 1, pp. 172-178, 949-982 (1995).

“Inhibition,” in the context of enzymes, refers to reversible enzyme inhibition such as competitive, uncompetitive and non-competitive inhibition. Competitive, uncompetitive and non-competitive inhibition can be distinguished by the effects of an inhibitor on the reaction kinetics of an enzyme. Competitive inhibition occurs when the inhibitor combines reversibly with the enzyme in such a way that it competes with a normal substrate for binding at the active site. The affinity between the inhibitor and the enzyme may be measured by the inhibitor constant, Kᵢ, which is defined as:

$${K}_{i} = \frac{[ E ] [ I ]}{[ EI ]}$$

wherein [E] is the concentration of the enzyme, [I] is the concentration of the inhibitor, and [EI] is the concentration of the enzyme-inhibitor complex formed by the reaction of the enzyme with the inhibitor. Unless otherwise specified, Kᵢ as used herein refers to the affinity between the inventive compounds and NAALADase. “C₅₀” is a related term used to define the concentration or amount of a compound that is required to cause a 50% inhibition of the target enzyme.

“NAALADase inhibitor” refers to any compound that inhibits NAALADase enzyme activity. Preferably, a NAALADase inhibitor exhibits a Kᵢ of less than 100 μM, more preferably less than 10 μM, and even more preferably less than 1 μM, as determined using any appropriate assay known in the art.

“Isomers” refer to compounds having the same number and kind of atoms, and hence the same molecular weight, but differing in respect to the arrangement or configuration of the atoms.

“Optical isomers” refer to enantiomers or diastereoisomers.

“Stereoisomers” are isomers that differ only in the arrangement of the atoms in space.

“Diastereoisomers” are stereoisomers that are not mirror images of each other. Diastereoisomers occur in compounds having two or more asymmetric carbon atoms; thus, such compounds have 2ⁿ optical isomers, where n is the number of asymmetric carbon atoms.

“Enantiomers” are a pair of stereoisomers that are non-superimposable mirror images of each other. Enantiomers result, for example, from the presence of one or more asymmetric carbon atom(s) in the compound (e.g., glyceraldehyde, lactic acid, sugars, tartaric acid, amino acids).

“Enantiomer-enriched” refers to a mixture in which one enantiomer predominates.

“Racemic mixture” means a mixture containing equal amounts of enantiomers.

“Non-racemic mixture” is a mixture containing unequal amounts of enantiomers.

“Animal” refers to a living organism having sensation and the power of voluntary movement, and which requires for its existence oxygen and organic food. Examples include, without limitation, members of the human, equine, porcine, bovine, murine, canine, or feline species. In the case of a human, an “animal” may also be referred to as a “patient”.

“Mammal” refers to a warm-blooded vertebrate animal.

“Treating ALS” refers to:

(i) delaying onset of ALS or ALS symptom(s);

(ii) slowing progression of ALS or ALS symptom(s);

(iii) prolonging survival of an animal suffering from ALS; and/or

(iv) attenuating ALS symptom(s).

In addition, “treating ALS” may optionally include:

(i) preventing ALS from occurring in an animal that may be predisposed to ALS but has not yet been diagnosed as having it;

(ii) inhibiting ALS, e.g. arresting its development; and/or

(iii) relieving ALS, e.g. causing regression of the disease, disorder and/or condition.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

### METHODS OF THE PRESENT INVENTION

The present invention relates to a method of treating amyotrophic lateral sclerosis (“ALS” ) comprising administering an effective amount of a NAALADase inhibitor to an animal in need of such treatment.

In a preferred embodiment, treating ALS is delaying onset of ALS or ALS symptom(s).

In another preferred embodiment, treating ALS is slowing progression of ALS or ALS symptom(s).

In another preferred embodiment, treating ALS is prolonging survival of an animal suffering from ALS.

In another preferred embodiment, treating ALS is attenuating one or more ALS symptom(s). ALS symptoms include without limitation muscular weakness and atrophy (particularly in the hands and feet), anterior horn dysfunction (particularly in the hands and feet), cramps, muscle twitches (fasciculations), spasticity, hyperactive deep tendon reflexes, extensor plantar reflexes, corticospinal tract degeneration, dysarthria and dysphagia.

### Pharmaceutical Compositions of the Present Invention

The present invention further relates to a pharmaceutical composition comprising:

(i) an effective amount of a NAALADase inhibitor for treating ALS in an animal; and

(ii) a pharmaceutically acceptable carrier.

### NAALAdase Inhibitors

NAALADase inhibitors that can be used in the inventive methods and pharmaceutical compositions include without limitation metallopeptidase inhibitors such as o-phenanthroline, metal chelators such as EGTA and EDTA, and peptide analogs such as quisqualic acid and β-NAAG.

While the pathophysiology of ALS is not well understood, there is evidence that it may involve glutamate excitotoxicity. Rothstein, J. D. et al., Ann. Neurol. (July 1990) 28(1):18-25; Tsai, G. et al., Brain Research (Dec. 3, 1993) 629(2):305-9. Thus, a preferred NAALADase inhibitor is one that is capable of reducing or preventing glutamate-induced excitotoxicity, preferably by altering glutamate release or biosynthesis presynaptically. While the foregoing attributes are preferred, the NAALADase inhibitors used in the inventive methods and pharmaceutical compositions may exert their therapeutic effects through other mechanisms of action.

Another preferred NAALADase inhibitor is an acid containing a metal binding group.

### Formula I

Another preferred NAALADase inhibitor is a compound of formula I:

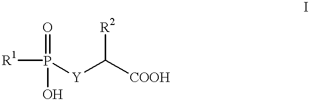

or an enantiomer or a pharmaceutically acceptable equivalent of said compound, wherein:

Y is CR³R⁴, NR⁵ or O;

R¹ is hydrogen, C₁-C₉ alkyl, C₂-C₉ alkenyl, C₃-C₈ cycloalkyl, C₅-C₇ cycloalkenyl, Ar, COOR⁶, NR⁶R⁷ or OR⁶, wherein said alkyl, alkenyl, cycloalkyl and cycloalkenyl are independently unsubstituted or substituted with one or more substituent(s), preferably, independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, COOR⁶, NR⁶R⁷ and Ar;

R² is hydrogen, C₁-C₆ alkyl, C₂-C₆ alkenyl, C₃-C₈ cycloalkyl, C₁-C₇ cycloalkenyl, Ar, halo or carboxy, wherein said alkyl, alkenyl, cycloalkyl and cycloalkenyl are independently unsubstituted or substituted with one or more substituent(s), preferably, independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, NR⁶R⁷ and Ar;

R³ and R⁴ are independently hydrogen or C₁-C₃ alkyl;

R⁵ is hydrogen or C₁-C₃ alkyl;

R⁶ and R⁷ are independently hydrogen, C₁-C₉ alkyl, C₂-C₉ alkenyl, C₃-C₈ cycloalkyl, CS—C₇ cycloalkenyl or Ar, wherein said alkyl, alkenyl, cycloalkyl and cycloalkenyl are independently unsubstituted or substituted with one or more substituent s), preferably, independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy and Ar; and

Ar is selected from the group consisting of 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl and phenyl, wherein said Ar is unsubstituted or substituted with one or more substituent(s), preferably, independently selected from the group consisting of halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, carboxy and N⁶R⁷.

In one embodiment of formula I, Y is CH₂.

In another embodiment, R² is —(CH₂)₂COOH.

In a further embodiment, R¹ is hydrogen, C₁-C₄ alkyl, C₂-C₄ alkenyl, C₃-C₈ cycloalkyl, C₅-C₇ cycloalkenyl, benzyl, phenyl or OR⁶, wherein said alkyl, alkenyl, cycloalkyl, cycloalkenyl, benzyl and phenyl are independently unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, NR⁶R⁷, benzyl and phenyl.

Preferred compounds of formula I are selected from the group consisting of:

2-(phosphonomethyl)pentanedioic acid;

2-[[(2-carboxyethyl)hydroxyphosphinyl]methyl]-pentanedioic acid;

2-[(benzylhydroxyphosphinyl)methyl]pentanedioic acid;

2-[(phenylhydroxyphosphinyl)methyl]pentanedioic acid;

2-[[((hydroxy)phenylmethyl)hydroxyphosphinyl]-methyl]pentanedioic acid;

2-[(butylhydroxyphosphinyl)methyl]pentanedioic acid;

2-[[(3-methylbenzyl)hydroxyphosphinyl]methyl]-pentanedioic acid;

2-[(3-phenylpropylhydroxyphosphinyl)methyl]-pentanedioic acid;

2-[[(4-fluorophenyl)hydroxyphosphinyl]methyl]-pentanedioic acid;

2-[(methylhydroxyphosphinyl)methyl]pentanedioic acid;

2-[(phenylethylhydroxyphosphinyl)methyl]pentanedioic acid;

2-[[(4-methylbenzyl)hydroxyphosphinyl]methyl]-pentanedioic acid;

2-[[(4-fluorobenzyl)hydroxyphosphinyl]methyl]-pentanedioic acid;

2-[[(4-methoxybenzyl)hydroxyphosphinyl]methyl]-pentanedioic acid;

2-[[(3-trifluoromethylbenzyl)hydroxyphosphinyl]-methyl]pentanedioic acid;

2-[[4-trifluoromethylbenzyl)hydroxyphosphinyl]-methyl]pentanedioic acid;

2-[[(2-fluorobenzyl)hydroxyphosphinyl]methyl]-pentanedioic acid;

2-[[(2,3,4,5,6-pentafluorobenzyl)hydroxy-phosphinyl]methyl]pentanedioic acid; and

enantiomers and pharmaceutically acceptable equivalents.

### Formula II

Another preferred NAALADase inhibitor is a compound of formula II

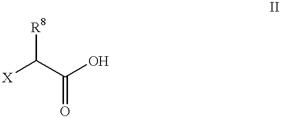

or an enantiomer or a pharmaceutically acceptable equivalent of said compound, wherein:

X is a moiety of formula III, IV or V

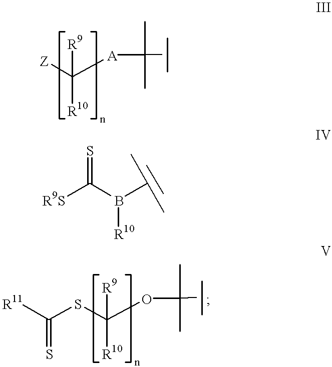

Z is SH, SO_{3H}, SO₂H, SOH, SO (NH) R¹² or S(NHR ²)₂R¹³;

B is N or CR¹⁴;

A is O, S, CR¹⁵R¹⁶ or (CR¹⁵R¹⁶) mS;

m and n are independently 0, 1, 2, 3 or 4;

R⁸, R⁹, R¹⁰, R¹¹, R¹², R₁₄, R¹⁵ and R¹⁶ are independently hydrogen, C₁-C₉ alkyl, C₂-C₉ alkenyl, C₃-C₈ cycloalkyl, C₅-C₇ cycloalkenyl, Ar¹, hydroxy, carboxy, carbonyl, amino, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and

Ar¹ is a carbocyclic or heterocyclic moiety, which is unsubstituted or substituted with one or more substituent(s);

provided that when X is a moiety of formula III and A is 0, then n is 2, 3 or 4; when X is a moiety of formula III and A is S, then n is 2, 3 or 4; and when X is a moiety of formula III and A is (CR¹⁵R¹⁶)mS, then n is 0, 2, 3 or 4.

In one embodiment of formula II, X is a moiety of formula III; n is 0, 1, 2 or 3; Z is SH, SO₃H, SO₂H, SOH or S(NHR¹²)₂R¹³; and A is O, S or CR¹⁵R¹⁶.

In another embodiment, R⁸ is —(CH₂)₂COOH.

In a further embodiment, Z is SH.

Preferred compounds of formula II are selected from the group consisting of:

2-(2-sulfanylethyl)pentanedioic acid;

3-(2-sulfanylethyl)-1,3,5-pentanetricarboxylic acid;

2-(2-sulfanylpropyl)pentanedioic acid;

2-(2-sulfanylbutyl)pentanedioic acid;

2-(2-sulfanyl-2-phenylethyl)pentanedioic acid;

2-(2-sulfanylhexyl)pentanedioic acid;

2-(2-sulfanyl-1-methylethyl)pentanedioic acid;

2-[1-(sulfanylmethyl)propyl]pentanedioic acid;

2-(3-sulfanylpentyl)pentanedioic acid;

2-(3-sulfanylpropyl)pentanedioic acid;

2-(3-sulfanyl-2-methylpropyl)pentanedioic acid;

2-(3-sulfanyl-2-phenylpropyl)pentanedioic acid;

2-(3-sulfanylbutyl)pentanedioic acid;

2-[3-sulfanyl-2-(phenylmethyl)propyl]pentanedioic acid;

2-[2-(sulfanylmethyl)butyl]pentanedioic acid;

2-[2-(sulfanylmethyl)pentyl]pentanedioic acid;

2-(3-sulfanyl-4-methylpentyl)pentanedioic acid; and

enantiomers and pharmaceutically acceptable equivalents.

### Formula VI

Another preferred NAALADase inhibitor is a compound of formula VI

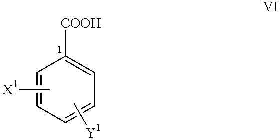

or an enantiomer or a pharmaceutically acceptable equivalent of said compound, wherein:

X¹ is —W—Z¹;

W is a bond or a linking group;

Z¹ is a terminal group; and

Y¹ is —COOH oriented meta or para relative to C-1.

Linking groups include, without limitation, divalent hydrocarbon chains, ethers, sulfides and amines, wherein the hydrocarbon chain, whether alone or part of the ether, sulfide or amine, may be saturated or unsaturated, straight or branched, open or closed, unsubstituted or substituted with one or more substituent(s), preferably, independently selected from the group consisting of C₁-C₆ alkoxy, C₂-C₆ alkenyloxy, phenoxy, benzyloxy, hydroxy, carboxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitro, nitroso, isonitroso, nitrosamino, imino, nitrilo, isonitrilo, nitrosimino, oxo, C₁-C₆ alkylthio, sulfamino, sulfamoyl, sulfeno, sulfhydryl, sulfinyl, sulfo, sulfonyl, sulfoxy, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, diselanyl, siloxy, silyl and silylene.

Preferably, W is a bond, —(CR¹⁷R¹⁸)ₙ—, —(CR¹⁷R¹⁸)ₙO(CR¹⁹R²⁰) ₘ—, —(CR¹⁷R¹⁸)ₙS(CR¹⁹R²⁰)ₘ— or —(CR¹⁷R¹⁸)ₙNR²¹(CR¹⁹R²⁰) ₘ—, wherein m and n are independently 0-9, and R¹⁷, R¹⁸, R¹⁹, R²⁰ and R²¹ are independently hydrogen, C₁-C₆ alkyl, C₂-C₆ alkenyl, C₂-C₆ alkynyl, C₆-C₁₄ aryl, heteroaryl, C₆-C₁₄ carbocycle, heterocycle, halo, hydroxy, sulfhydryl, nitro, amino or C₁-C₆ alkoxy, and said alkyl, alkenyl, alkynyl, aryl, heteroaryl, carbocycle, heterocycle and alkoxy are independently unsubstituted or substituted with one or more substituent(s). More preferably, R¹⁷, R¹⁸, R¹⁹, R²⁰ and R²¹ are each hydrogen and the total number of carbon atoms in W is 2-6.

Preferably, Z¹ is a metal binding group. More preferably, Z¹ is —COOH, —COR²², —OR²², —CF₃, —CN, —F, —Cl, —Br, —I, —NO, —NO₂, —C(O)(NR²²OR²³), —C(O)(NR²²PO₃H₂), —C(O)(NR²²R²³), ═NOH, —NR²²(P(O)(R²³)OH), ═NR²², —N═NR²², —N(R²²)CN, —NR²²(CR²³R²⁴)ₚCOOH, —NR²²(CO)NR²³R²⁴, —NR²²(COOR²³), —NR²²(CO)R²³, —NR²²(OR²³), —NR²²R²³, —NR²²(SO₂R²³), —O(CO)R²², —OR²², —SO₂(OR²²), —SO₂(NR²²R²³), —SO₂R²², —SO₃R²², —SNR²²(OR²³), —S(NR²²R²³), —SR²², —SSR²², —P(O)(OH)OR²², —P(O)(OH) R²² or —PR²²R²³, wherein p is 0-6, and R²², R²³ and R²⁴ are independently hydrogen, C₁-C₉ alkyl, C₂-C₉ alkenyl, C₂-C₉ alkynyl, C₆-C₁₄ aryl, heteroaryl, C₆-C₁₄ carbocycle, heterocycle, halo, hydroxy, sulfhydryl, nitro, amino or C₁-C₉ alkoxy, and said alkyl, alkenyl, alkynyl, aryl, heteroaryl, carbocycle, heterocycle and alkoxy are independently unsubstituted or substituted with one or more substituent(s). Even more preferably, Z¹ is —NH (CR²³ R²⁴)ₚCOOH, —PO(OH)OR²², —PO(OH)R²², —NR²²(P(O)(R²³)OH), —CON(R²²)(OH) or —SH.

In one embodiment of formula VI:

X¹ is —(CR¹⁷R¹⁸)ₙNH (CR¹⁹R²⁰)ₘCOOH, —PO(OH)OR²², —(CR¹⁷R¹⁸)ₙP(O)(OH) R²², —NH—(CR¹⁹R²⁰)ₘ-heteroaryl, —NH (P(O)(R²³) OH)—, —(CR¹⁷R¹⁸)NH(P(O)(OH)R²³), —CON(R²²)(OH)—(CR¹⁷CR¹⁸)ₙCON (R²²)(OH), —(CR¹⁷R¹⁸)ₙSH or —O(CR¹⁹R²⁰)ₘSH, —SO₂NH-aryl, —N(C═O)—CH₂(C═O)-aryl, —SO₂NH-aryl, —N(C═O)—CH₂(C═O)-aryl, —O-aryl wherein aryl in —O-aryl is substituted by at least one of nitro, carboxy or

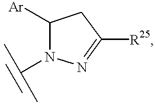

wherein X¹ is oriented meta or para relative to C-1;

m and n are independently 1-3, provided that when X¹ is —O(CR¹⁹R²⁰)ₘSH, then m is 2 or 3;

R¹⁷, R¹⁸, R¹⁹, R²⁰, R²², R²³ and R²⁵ are independently hydrogen, C₁-C₆ alkyl, C₂-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituent (s); and

Y¹ is —COOH oriented meta or para relative to C-1.

Preferably, when X is —PO(OH)OR²² or —(CR¹⁷R¹⁸)ₙP(O)(OH)OR²², then R²² is not H or methyl; when X is —NH (P(O)R²³)OH or —(CR¹⁷ R¹⁸)ₙNH(P(O)(OH)R²³), then R²³ is not benzyl unsubstituted or substituted with amino; and when X is —CON(R²²)(OH), then R²² is not H or methyl.

In another embodiment of formula VI, X¹ is oriented meta relative to C-1, and Y¹ is oriented ortho relative to X¹ and para relative to C-1. Preferably, W is a bond, —(CH₂)ₙ—NH—(CH₂)ₘ— or —(CH₂)ₙ—; m is 1-3; n is 0-3; and Z¹ is —CO₂H, —NO₂,—NH₂, —SO₃H, halo, C₅-C₆ heteroaryl, carboxyphenylthio, or mono- or di-carboxyphenylsulfonyl.

Examples of this embodiment are:

2-[(4-carboxyphenyl)sulfonyl]-1,4-benzene-dicarboxylic acid;

2-[(2,5-dicarboxyphenyl) sulfonyl]-1,4-benzene-dicarboxylic acid;

1,2,4-benzenetricarboxylic acid;

2-[(2-carboxyphenyl)thiol-1,4-benzenedicarboxylic acid;

2-nitro-1,4-benzenedicarboxylic acid;

2-bromo-1,4-benzenedicarboxylic acid;

2-amino-1,4-benzenedicarboxylic acid;

2-sulfoterephthalic acid, monosodium salt;

2-carboxymethyl-1,4-benzenedicarboxylic acid;

2-[(2-furanylmethyl)-amino]-1,4-benzenedicarboxylic acid;

2-[(carboxymethyl)amino]-1,4-benzenedicarb oxylic acid; and

enantiomers and pharmaceutically acceptable equivalents.

In another embodiment of formula VI, X¹ is oriented ortho relative to C-1, and Y¹ is oriented para relative to X1 and meta relative to C-1. Preferably, (1) when W is a bond, then Z¹ is —CO₂H, —OH, —NO₂, —C(O)(NHR²³), —SR²³, —COR²³ or —NH(CH₂R²³), and R²³ is an aryl or a heteroaryl wherein said aryl and heteroaryl are independently unsubstituted or substituted with one or more alkyl, nitro or carboxy group(s); and (2) when W is —(CH₂)ₙ— and n is 1-3, then Z¹ is —SH.

Examples of this embodiment are:

4-(4-nitrobenzoyl)-1,3-benzenedicarboxylic acid;

4-[4-(2,4-dicarboxybenzoyl)phenoxy]-1,2-benzene-dicarboxylic acid;

4-[[(2,4,6-trimethylphenyl)amino]carbonyl]-1,3-benzenedicarboxylic acid;

4-nitro-1,3-benzenedicarboxylic acid;

4-[(1-naphthalenylamino)-carbonyl]-1,3-benzene-dicarboxylic acid;

1,2,4-benzenetricarboxylic acid;

4-[(2-carboxyphenyl)thio]-1,3-benzenedicarboxylic acid;

4-[3-[[3-(2,4-dicarboxyphenoxy)propyl]dithio]-propoxy]-1,3-benzenedicarboxylic acid;

4-hydroxy-1,3-benzenedicarboxylic acid;

4-[(2-furanylmethyl)amino]-1,3-benzenedicarboxylic acid;

4-(2-mercaptoethyl)-1,3-benzenedicarboxylic acid; and

enantiomers and pharmaceutically acceptable equivalents.

In another embodiment of formula VI, X¹ is oriented meta relative to C-1, and Y¹ is oriented meta relative to X and meta relative to C-1. Preferably, (1) when W is a bond, —(CH₂)ₙ— or —O(CH₂)ₘ— and m and n are independently 0-3, then Z¹ is —SO₃H, —NO₂, —NH₂, —CO₂H, —OH, —PO₃H, —CO(NHOH) or —SH; (2) when W is —(CH₂)ₙNH(CH₂)ₘ— and m and n are independently 0-3, then Z¹ is —CO₂H or C₅-C₆ heteroaryl; and (3) when W is a bond, then Z¹ is either (a) a heteroaryl that is unsubstituted or substituted with an aryl that is unsubstituted or substituted with one or more C₁-C₃ alkyl, halo, nitro or hydroxy group(s), or (b) —SO2(NHR²⁴) or —NH(COR²⁴), wherein R²⁴ is an aryl that is unsubstituted or substituted with one or more nitro, amino, halo or hydroxy group(s).

Examples of this embodiment are:

5-[4,5-dihydro-5-(4-hydroxyphenyl)-3-phenyl-1H-pyrazol-1-yl]-1,3-benzenedicarboxylic acid;

5-(4,5-dihydro-3-methyl-5-phenyl-1H-pyrazol-1-yl)-1,3-benzenedicarboxylic acid;

5-([(4-chloro-3-nitrophenyl)amino]sulfonyl]-1,3-benzenedicarboxylic acid;

5-[[[4-chloro-3-[[3-(2-methoxyphenyl)-1,3-dioxopropyl]amino]phenyl]amino]sulfonyl-1,3-benzenedicarboxylic acid;

5-[[3-[4-(acetylamino)phenyl]-1,3-dioxopropyl]amino]-1,3-benzenedicarboxylic acid;

5-acetylamino-1,3-benzenedicarboxylic acid;

5-[[(1-hydroxy-2-naphthalenyl)carbonyl]-methylamino]-1,3-benzenedicarboxylic acid;

5-(4-carboxy-2-nitrophenoxy)-1,3-benzenedicarboxylic acid;

5-sulfo-1,3-benzenedicarboxylic acid;

5-nitro-1,3-benzenedicarboxylic acid;

5-amino-1,3-benzenedicarboxylic acid;

1,3,5-benzenetricarboxylic acid;

5-[[(3-amino-4-chlorophenyl)amino]sulfonyl]-1,3-benzenedicarboxylic acid;

5-(3-mercaptopropoxy)-1,3-benzenedicarboxylic acid;

5-hydroxy-1,3-benzenedicarboxylic acid;

5-(2-mercaptoethoxy)-1,3-benzenedicarboxylic acid;

5-[(hydroxyamino)carbonyl]-1,3-benzenedicarboxylic acid;

5-phosphono-1,3-benzenedicarboxylic acid;

5-mercaptomethyl-1,3-benzenedicarboxylic acid;

5-phosphonomethyl-1,3-benzenedicarboxylic acid;

5-[[(carboxymethyl)amino]-methyl]-1,3-benzene-dicarboxylic acid;

5-[(carboxymethyl)amino]-1,3-benzenedicarboxylic acid;

5-[[(2-furanylmethyl)amino]-methyl]-1,3-benzene-dicarboxylic acid;

5-[2-(hydroxyamino)-2-oxoethyl]-1,3-benzene-dicarboxylic acid;

5-(2-mercaptoethyl)-1,3-benzenedicarboxylic acid; and

enantiomers and pharmaceutically acceptable equivalents.

### Other NAALADase Inhibitors

Other NAALADase inhibitors are described in International Publication No. WO 01/14390 and copending U.S. patent application Ser. No. 09/438,970 filed Nov. 12, 1999 (corresponding to International Patent Application No. PCT/US00/30977 filed Nov. 13, 2000), the entire contents of which publication and applications are herein incorporated by reference as though set forth herein in full.

Possible substituents of the compounds of formulas I-VI include, without limitation, C₁-C₆ alkyl, C₂-C₆ alkenyl, C₂-C₆ alkynyl, C₁-C₆ alkoxy, C₂-C₆ alkenyloxy, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, disilanyl, siloxy, silyl, silylene and carbocyclic and heterocyclic moieties.

Carbocyclic moieties include alicyclic and aromatic structures. Examples of carbocyclic and heterocyclic moieties include, without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, and phenoxazinyl.

All variables of formulas I-VI are independently selected at each occurrence. For example, formula II may have two different CR10R¹¹ moieties when X is a moiety of formula III and n is 2, with the first CR10R¹¹ moiety being CH₂, and the second CR¹⁰R¹¹ moiety being CH(CH₃).

The compounds of formulas I-VI may possess one or more asymmetric carbon center(s) and, thus, may be capable of existing in the form of optical isomers as well as in the form of racemic or non-racemic mixtures of optical isomers. The optical isomers can be obtained by resolution of the racemic mixtures according to conventional processes well known in the art, for example by formation of diastereoisomeric salts by treatment with an optically active acid or base, and then separation of the mixture of diastereoisomers by crystallization followed by liberation of the optically active bases from these salts. Examples of optically active acids are tartaric, diacetyltartaric, dibenzoyltartaric, ditoluoyltartaric and camphorsulfonic acid. A different process for separation of optical isomers involves the use of a chiral chromatography column optimally chosen to maximize the separation of the enantiomers. Still another available method involves synthesis of covalent diastereoisomeric molecules, for example, esters, amides, acetals, ketals, and the like, by reacting compounds used in the inventive methods and pharmaceutical compositions with an optically active acid in an activated form, an optically active diol or an optically active isocyanate. The synthesized diastereoisomers can be separated by conventional means such as chromatography, distillation, crystallization or sublimation, and then hydrolyzed to deliver the enantiomerically pure compound. In some cases hydrolysis to the parent optically active drug is not necessary prior to dosing the patient since the compound can behave as a prodrug. The optically active compounds can likewise be obtained by utilizing optically active starting materials.

It is understood that the compounds of formulas I-VI encompass optical isomers as well as racemic and non-racemic mixtures.

### Synthesis of NAALADase Inhibitors

Some of the NAALADase inhibitors used in the inventive methods and pharmaceutical compositions can be readily prepared by standard techniques of organic chemistry, utilizing the general synthetic pathways and examples depicted in U.S. Pat. Nos. 5,672,592, 5,795,877, 5,863,536, 5,880,112, 5,902,817, 5,962,521, 5,968,915, 6,025,344, 6,025,345, 6,028,216, 6,046,180, 6,054,444, 6,071,965 and 6,121,252, allowed U.S. patent application Ser. No. 09/228,391 for which the issue fee has been paid, copending U.S. patent application Ser. No. 09/438,970 filed Nov. 12, 1999 (corresponding to International Patent Application No. PCT/US00/30977 filed Nov. 13, 2000), and International Publications Nos. WO 99/33849, Wo 00/01668 and WO 01/14390, the entire contents of which patents, patent application and publications are herein incorporated by reference, as though set forth herein in full.

Other NAALADase inhibitors may be available from commercial suppliers or can be readily prepared by an ordinarily skilled artisan using standard techniques such as those disclosed in U.S. Pat. No. 5,859,046, the entire contents of which reference are herein incorporated by reference as though set forth herein in full.

Yet other NAALADase inhibitors can be readily prepared by standard techniques of organic chemistry, utilizing the general synthetic pathways depicted below in SCHEMES I-VI.

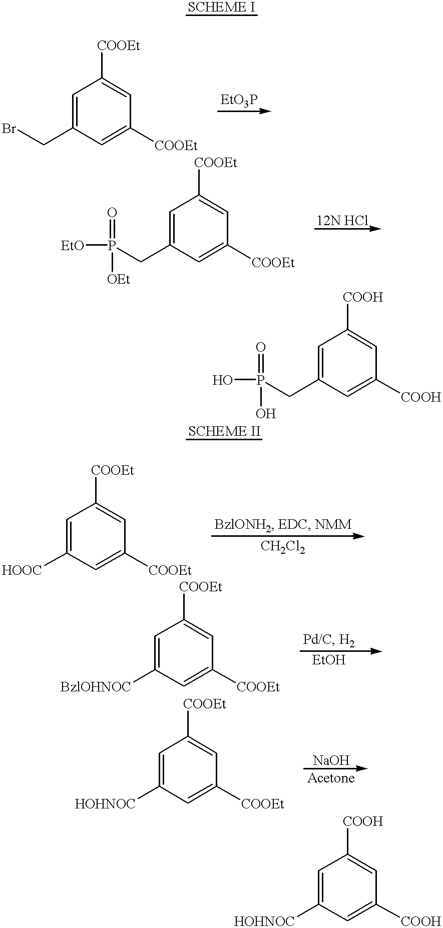

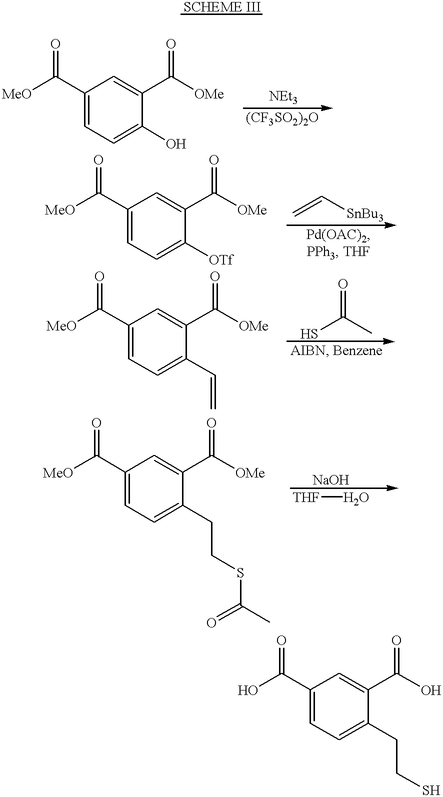

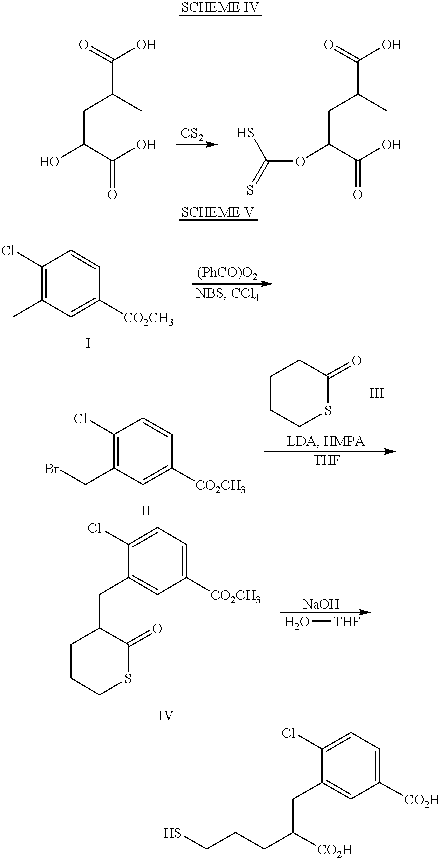

### Route of Administration

In the inventive methods, the compounds will generally be administered to a patient in the form of a pharmaceutical formulation. Such formulation preferably includes, in addition to the active agent, a physiologically acceptable carrier and/or diluent. The compounds may be administered locally or systemically by any means known to an ordinarily skilled artisan. For example, the compounds may be administered orally, parenterally, by inhalation spray, topically, rectally, nasally, buccally, vaginally or via an implanted reservoir in dosage formulations containing conventional non-toxic pharmaceutically acceptable carriers, adjuvants and vehicles. The term parenteral as used herein includes subcutaneous, intravenous, intraarterial, intramuscular, intraperitoneal, intrathecal, intraventricular, intrasternal, intracranial or intraosseous injection and infusion techniques. The exact administration protocol will vary depending upon various factors including the age, body weight, general health, sex and diet of the patient; the determination of specific administration procedures would be routine to an ordinarily skilled artisan.

Preferably, the compounds and compositions used in the inventive methods are capable of crossing the blood-brain barrier. Compounds and compositions that do not freely cross the blood-brain barrier may be administered by an intraventricular route or by other methods recognized in the art.

### Dosage

In the inventive methods, the compounds and compositions may be administered by a single dose, multiple discrete doses or continuous infusion. Pump means, particularly subcutaneous pump means, are preferred for continuous infusion.

Dose levels on the order of about 0.001 to about 10,000 mg/kg of the active ingredient compound are useful in the inventive methods, with preferred levels being about 0.1 to about 1,000 mg/kg, and more preferred levels being about 1 to 100 mg/kg. The specific dose level for any particular patient will vary depending upon a variety of factors, including the activity and the possible toxicity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

### Administration Regimen

For the inventive methods, any administration regimen well known to an ordinarily skilled artisan for regulating the timing and sequence of drug delivery can be used and repeated as necessary to effect treatment. Such regimen may include pretreatment and/or co-administration with additional therapeutic agents.

### Co-administration with Other Treatments

In the inventive methods, the NAALADase inhibitors and pharmaceutical compositions may be used alone or in combination with one or more additional agent(s) for simultaneous, separate or sequential use.

The additional agent(s) may be any therapeutic agent(s) known to an ordinarily skilled artisan, including, without limitation, (an)other compound(s) of formulas I-VI.

The NAALADase inhibitors and pharmaceutical compositions may be co-administered with one or more therapeutic agent(s) either (i) together in a single formulation, or (ii) separately in individual formulations designed for optimal release rates of their respective active agent. Each formulation may contain from about 0.01% to about 99.99% by weight of a NAALADase inhibitor, as well as one or more pharmaceutically acceptable carrier(s), such as wetting, emulsifying and/or pH buffering agent(s).

In addition, the NAALADase inhibitors and pharmaceutical compositions may be administered prior to, during or following surgery or physical therapy.

### EXAMPLES

The following examples are illustrative of the present invention and are not intended to be limitations thereon. Unless otherwise indicated, all percentages are based upon 100% by weight of the final composition.

### Example 1

### Preparation of 5-phosphonomethyl-1,3-benzenedicarboxylic acid (SCHEME I)

Diethyl 5-[(diethoxyphosphinyl)methyl]-1,3-benzenedicarboxylate

A solution of 5-bromomethyl-1,3-benzenedicarboxylate (Collman et al., J. Am. Chem. Soc., 116(14) (1994) 6245-6251; 0.315 g, 1.0 mmol) in triethylphosphite (3.0 mL) was heated at 150° C. for 5 hours. The solvent was removed under reduced pressure and the residual oil was purified by chromatography to give 0.248 g of colorless oil: ¹H NMR (CDCl₃) δ 1.28 (t, 3H), 1.42 (t, 3H), 3.26 (d, 2H), 4.06 (q, 2H), 4.41 (q, 2H), 8.17 (s, 2H), 8.58 (s, 1H). TLC: R_{f} 0.10 (EtOAc/Hexanes 1/1).

5-Phosphonomethyl-1,3-benzenedicarboxylic acid

A solution of diethyl 5-[(diethoxyphosphinyl)methyl]-1,3-benzenedicarboxylate (0.186 g, 0.5 mmol) in 12 N HCl (2.5 mL) was heated at 100° C. for 24 hours. The resulting precipitate was washed with water and dried under vacuum to give 0.057 g of white powder: ¹H NMR (D₂O) δ 3.11 (d, 2H), 7.93 (s, 2H), 8.19 (s, 1H). TLC: R_{f} 0.20 (EtOAc/Hexanes 1/1). Elemental analysis calculated for C₉H₇O₇P.H₂O: C, 38.86; H, 3.99. Found: C, 38.74; H, 4.08.

### Example 2

### Preparation of 5-[(hydroxyamino)carbonyl]-1,3-benzene-dicarboxylic acid (SCHEME II)

Diethyl 5-[[(phenylmethoxy)amino]carbonyl]-1,3-benzenedicarboxylate

To a solution of diethyl 1,3,5-benzenetricarboxylate (3.192 g, 20 mol) and O-benzylhydroxyamine hydrochloride (4.789 g, 19 mmol) in 40 mL were added N-methylmorpholine (2.2 mL, 20 mmol) and EDC (3.834 g, 20 mmol) at 0° C., and the mixture was stirred at room temperature for 20 hours. The solvent was removed by evaporator and the residue was dissolved in EtOAc (150 mL). The organic solution was washed with 1 N HCL (150 mL), washed with saturated aqueous NaHCO₃ (50 mL), dried over Na₂SO₄, and concentrated to give white solid. This material was recrystallized from EtOAc to give 4.154 g of white powder: ¹H NMR (CDCl₃) δ 1.41 (t, 6H), 4.40 (q, 4H), 5.05 (s, 2H), 7.3-7.5 (m, 5H), 8.52 (s, 2H), 8.76 (s, 1H), 9.1 (br, 1H). TLC: R_{f} 0.62 (EtOAc/Hexanes 1/1).

Diethyl 5-[(hydroxyamino)carbonyl]-1,3-benzenedicarboxylate

To a solution of diethyl 5-[[(phenylmethoxy)amino]-carbonyl]-1,3-benzenedicarboxylate (0.742 g, 2.0 mmol) in ethanol (10 mL) was added a suspension of Pd/C in ethanol (5 mL), and the mixture was shaken under hydrogen (50 psi) for 20 hours. The catalyst was removed by filtration through a pad of celite and the filtrate was concentrated to give white powder. This material was washed with ethanol (10 mL×2) and dried under vacuum to give 0.380 g of white powder: ¹H NMR (CD₃OD) δ 1.44 (t, 6H), 4.45 (q, 4H), 8.60 (s, 2H), 8.72 (s, 1H). TLC: R_{f} 0.20 (EtOAc/Hexanes 1/1).

5-[(Hydroxyamino)carbonyl]-1,3-benzene-dicarboxylic acid

To a solution of diethyl 5- (hydroxyamino)carbonyl]-1,3-benzenedicarboxylate (0.281 g, 1.0 mmol) in acetone (5 mL) was added 1.0 N NaOH (5 mL) at room temperature, and the mixture was stirred at room temperature for 2 hours. The solvent was removed under reduced pressure and the residue was taken up with 1 N HCl (15 mL) to give white precipitate. This material was dried under vacuum to give 0.096 g of white solid: ¹H NMR (D₂O) δ 8.52 (s, 2H), 8.76 (s, 1H). Elemental analysis calculated for C₉H₇NO₆.H₂O: C, 44.45; H, 3.73; N, 5.76. Found: C, 44.47; H, 3.78; N, 5.74.

### Example 3

### Preparation of 4-(2-mercaptoethyl)-1,3-benzenedicarboxylic acid (SCHEME III)

Dimethyl 4-trifluoromethanesulfonyloxy-1,3-benzenedicarboxylate

To a solution of dimethyl 4-hydroxy-isophthalate (0.850 g, 4.04 mmol) in CH₂Cl₂ (15 mL) were added triethylamine (0.6 mL, 4.3 mmol) and triflic anhydride (0.8 mL, 4.76 mmol) at 0° C., and the mixture was stirred at 0° C. for 18 hours. The solvent was evaporated and the residue was diluted with ether (30 mL). The organic solution was washed with 1 N HCl (30 mL×3), dried over MgSO₄, and concentrated to give 1.30 g of dark yellow oil (93% yield): ¹H NMR (CDCl₃) δ 3.97 (s, 3H), 4.00 (s, 3H) 7.4 (d, 1H), 8.3 (d, 1H), 8.74 (s, 1H)

Dimethyl 4-ethenyl-1,3-benzenedicarboxylate

To a solution of dimethyl 4-trifluoromethanesulfonyl-oxy-1,3-benzenedicarboxylate (1.5 g, 4.38 mmol) in dioxane (50 mL) were added Pd(PPh₃)₄ (510 mg, 0.44 mmol), lithium chloride (1.3 g, 30.7 mmol) and tributyl(vinyl)tin (1.5 mL, 5.13 mmol) at room temperature. The mixture was heated at 100° C. for 5 hours. The reaction mixture was filtered and the filtrate was concentrated and passed through a column of silica gel (Hexanes/EtOAc=10:1) to give 1.1 g of colorless oil (84% yield): ¹H NMR: (CDCL₃) δ 3.92 (s, 3H), 3.93 (s, 3H), 5.45 (d, 1H), 5.73 (d, 1H), 7.49 (m, 1H), 7.66 (d, 1H), 8.13 (d, 1H), 8.53 (s, 1H).

Dimethyl 4-[2- (acetylthio)ethyl]-1,3-benzenedicarboxylate

To a degassed solution of dimethyl 4-ethenyl-1,3-benzenedicarboxylate (415 mg, 1.88 mmol) in benzene (6 mL) were added AIBN (33 mg, 0.21 mmol) and thioacetic acid (0.27 mL, 3.78 mmol), and the mixture was refluxed for 5 hours. The reaction mixture was diluted with aqueous NaHCO₃ solution (15 mL) and extracted with EtOAc (15 mL). The organic layer was dried over MgSO₄ and concentrated. The residual material was purified by silica gel chromatography (hexanes/EtOAc=10:1) to give 0.150 g of colorless oil (27% yield): ¹H NMR (CDCl₃) δ 2.32 (s, 3H), 3.16 (t, 2H), 3.28 (t, 2H), 3.94 (s, 6H)δ 7.42 (d, 1H), 8.09 (d, 1H), 8.58 (s, 1H).

4-(2-Mercaptoethyl)-1,3-benzenedicarboxylic acid

To a degassed solution of dimethyl 4-[2-(acetylthio) ethyl]-1,3-benzenedicarboxylate (0.130 g, 0.44 mmol) in THF (5 mL) was added a degassed solution of 5 N NaOH (5 mL). The reaction mixture was stirred under nitrogen overnight. The reaction mixture was diluted with H₂O (10 mL) and extracted with EtOAC (10 mL). The organic layer was dried over MgSO₄ and concentrated to give 0.045 g of white solid (45% yield): ¹H NMR (DMSO) δ 2.67 (t, 2H), 3.21 (t, 2H), 7.37 (d, 1H), 7.98 (d, 1H), 8.46 (s, 1H). ¹³C NMR (DMSO) δ 26.64, 40.60, 130.87, 132.05, 133.46, 133.81, 134.13, 148.53, 169.22, 170.20. Elemental analysis calculated for C₁₀H₁₀SO₄: C, 53.09; H, 4.45; S, 14.47. Found: C, 53.37; H, 4.87; S, 12.84. MS(FAB): 225.

### Example 4

### Preparation of 5-carboxy-2-chloro-alpha-(3-mercaptopropyl)-benzenepropanoic acid (SCHEME V)

Methyl 3-bromomethyl-4-chlorobenzoate II

To a suspension of methyl 4-chloro-3-methylbenzoate I (19.9 g, 108 mmol) and N-bromosuccinimide (NBS, 20.2 g, 114 mmol) in carbon tetrachloride (500 mL) was added benzoyl peroxide (1.30 g, 5.4 mmol), and the mixture was stirred at 90° C. overnight. The mixture was then cooled and the white precipitate was removed by filtration. The filtrate was concentrated and the resulting solid was re-crystallized from ethyl acetate to give methyl 3-bromomethy-4-chlorobenzoate II (15.0 g, 57 mmol, 53%) as a white solid: ¹H NMR (CDCl₃) δ 3.95 (s, 3H), 4.63 (s, 2H), 7.49 (d, J=8.3 Hz, 1H), 7.94 (dd, J=2.1, 8.3 Hz, 1H), 8.15 (d, J=2.1 Hz, 1H)

3-(2-Chloro-5-methoxycarbonylbenzyl)-tetrahydrothiopyrane-2-one IV

To a solution of lithium diisopropylamide (2.0 M solution, 3.3 mL, 6.6 mmol) in THF (25 mL) was added tetrahydrothiopyran-2-one III (0.731 g, 6.3 mmol) at −40° C., and the mixture was stirred at −40° C. for 45 minutes. A solution of methyl 3-bromomethy-4-chlorobenzoate II (1.67 g, 6.3 mmol) in THF (10 mL) was then dropwise added to the mixture at −40° C. Subsequently, hexamethylphosphoramide (0.20 g, 1.4 mmol) was added to the mixture at −40° C., and the reaction mixture was stirred at −40° C. for 4 hours. A saturated ammonium chloride solution (30 mL) was added to the reaction mixture, and the organic solvent was removed under reduced pressure. The mixture was then partitioned between ether (150 mL) and H₂O (150 mL). The organic layer was washed with brine, dried over MgSO₄, and concentrated. The crude material was chromatographed on silica gel using EtOAc/hexanes to afford 3-(2-chloro-5-methoxycarbonylbenzyl)-tetrahydrothio-pyrane-2-one IV (0.60 g, 2.0 mmol, 32%) as a white solid: ¹H NMR (CDCl₃) δ 1.65-1.75 (m, 1H), 1.90-2.05 (m, 2H), 2.05-2.15 (m, 2H), 2.74 (dd, J=9.4, 13.9 Hz, 1H), 2.85-3.00 (m, 1H), 3.10-3.20 (m, 2H), 3.58 (dd, J=4.7, 13.9 Hz, 1H), 3.92 (s, 3H), 7.44 (d, J=8.3 Hz, 1H), 7.85 (dd, J=8.3, 2.1 Hz, 1H), 7.91 (d, J=2.1 Hz, 1H).

5-Carboxy-2-chloro-alpha-(3-mercaptopropyl) benzenepropanoic acid

A solution of 3-(2-Chloro-5-methoxycarbonylbenzyl)-tetrahydrothiopyrane-2-one IV (9.26 g, 31.0 mmol) in THF (70 mL) was purged for 15 minutes with nitrogen. A degassed aqueous sodium hydroxide solution (2.2 M, 70 mL, 154 mmol) was added to the solution and the mixture was stirred at room temperature under nitrogen overnight. The reaction mixture was washed with ether, acidified by 3N HCl at 0° C., and extracted with ether. The extract was dried over MgSO₄ and concentrated to afford 5-carboxy-2-chloro-alpha-(3-mercaptopropyl) benzenepropanoic acid (8.42 g, 27.8 mmol, 90%) as a white solid: ¹H NMR (CD₃OD) δ 1.50-1.80 (m, 4H), 2.35-2.50 (m, 2H), 2.65-2.75 (m, 1H), 2.91 (dd, J=6.2, 13.8 Hz, 1H), 2.96 (dd, J=8.8, 13.8 Hz, 1H), 7.39 (d, J=8.3 Hz, 1H), 7.76 (dd, J=2.0, 8.3 Hz, 1H), 7.86 (d, J=2.0 Hz, 1H); ³C NMR (CD₃OD) δ 25.1, 32.5, 33.2, 37.4, 46.8, 130.8, 131.2, 134.0, 139.1, 140.5, 169.2, 178.9. Elemental analysis calculated for C₁₃H₁₅ClO₄S: C, 51.57; H, 4.99; S, 10.59; Cl, 11.71. Found: C, 51.59; H, 4.94; S, 10.43; Cl, 11.80.

### Example 5

### Preparation of 3-carboxy-5-(1,1-dimethylethyl)-alpha-(3-mercaptopropyl)-benzenepropanoic acid (SCHEME VI)

Methyl 5-tert-butylhydrogenisophthalate VI

To a solution of dimethyl 5-tert-butylisophthalate V (23.0 g, 92 mmol) in methanol (150 mL) was added a solution of sodium hydroxide (3.68 g, 92 mmol) in H₂O (10 mL) at 25° C., and the mixture was stirred at 25° C. for 3 hours. The organic solvent was removed under reduced pressure and the residual solid was suspended in an aqueous sulfuric acid solution (1.0 M). The suspension was filtered and the precipitate was washed with H₂O , dried under vacuum, and crystallized from hexanes/ethyl acetate to afford methyl 5-tert-butylhydrogenisophthalate VI (16.3 g, 69.0 mmol, 75%) as a white solid: ¹H NMR (CDCl₃) δ 1.45 (s, 9H), 3.9 (s, 3H), 8.5 (s, 1H), 8.7 (s, 1H), 8.8 (s, 1H); ¹³C NMR (CDCl₃) δ 31.3 (3C), 35.2, 52.5, 128.8, 129.7, 130.7, 131.1, 131.6, 132.0, 166.7, 171.5.

Methyl 3-tert-butyl-5-hydroxymethylbenzoate VII

Borane-dimethyl sulfide complex (7.23 mL, 76.2 mmol) was slowly added to a solution of methyl 5-tert-butylhydrogenisophthalate VI (12.0 g, 50.8 mmol) in THF (100 ml) over the period of 20 minutes at room temperature. The mixture was stirred for 1.5 hours at room temperature and then refluxed for 1 additional hour. The reaction mixture was then cooled and the unreacted borane was decomposed with methanol (10 mL). The solvents were removed under reduced pressure and the residue was dissolved in ethyl acetate. The organic solution was washed with a saturated NaHCO₃ solution, dried over MgSo₄, and purified by a silica gel column chromatography (hexane/ethyl acetate) to afford methyl 3-tert-butyl-5-hydroxymethylbenzoate VII (10.0 g, 45.0 mmol, 90%) as a white solid: ¹H NMR (CDCl₃) δ 1.45 (s, 9H), 3.9 (s, 3H), 4.7 (s, 2H), 7.6 (s, 1H), 7.8 (s, 1H), 8.0 (s, 1H); ¹³C NMR (CDCl₃) δ 31.4 (3C), 35.0, 52.3, 65.3, 125.5, 126.1, 128.8, 130.3, 141.0, 152.1, 167.5.

Methyl 3-bromomethyl-5-tert-butylbenzoate VIII

To a solution of methyl 3-tert-butyl-5-hydroxymethylbenzoate VII (9.50 g, 42.7 mmol) and carbon tetrabromide (17.25 g, 52.0 mmol) in dichloromethane (50 mL) was slowly added triphenylphosphine (13.6 g, 52.0 mmol) over the period of 20 minutes, and the mixture was stirred at room temperature for 25 minutes. The reaction mixture was concentrated under reduced pressure and the residue was suspended in ethyl acetate. The precipitate was removed by filtration and the filtrate was concentrated. The crude material was purified by a silica gel chromatography (hexanes/ethyl acetate, 4:1), and the product was re-crystallized form ethyl acetate/hexanes to afford methyl 3-bromomethyl-5-tert-butylbenzoate VIII (12.0 g, 42.1 mmol, 99%) as a white solid: ¹H NMR (CDCl₃) δ 1.45 (s, 9H), 3.7 (s, 3H), 4.4 (s, 2H), 7.6 (s, 1H), 7.8 (s, 1H), 8.0 (s, 1H); ¹³C NMR (CDCl₃) δ 31.3 (3C), 33.2, 36.0, 52.3, 126.9, 127.5, 130.6, 130.7, 137.9, 152.4, 167.0.

5-(3-Tert-butyl-5-methoxycarbonyl-benzyl)-2,2-dimethyl-5-[3-[(triphenylmethyl)thio]propyl]-[1,3]dioxane-4,6-dione X

A solution of methyl 3-bromomethyl-5-tert-butylbenzoate (10.3 g, 36.1 mmol), 2,2-dimethyl-5-[3-[(triphenylmethyl)-thio]propyl]-[1,3]dioxane-4,6-dione IX (13.8 g, 30.0 mmol), and benzyltriethylammonium chloride (6.38 g, 30 mmol) in acetonitrile (90 mL) was added potassium carbonate (4.35 g, 30 mmol) at 25° C., and the reaction mixture was stirred at 60° C. overnight (the synthesis of compound IX was previously described in International Publication No. WO 00/01668). The solvent was removed under reduced pressure and the residue was partitioned between ethyl acetate and a 10% aqueous KHSO₄ solution. The organic layer was dried over MgSO₄, concentrated. The crude material was recrystallized from ethyl acetate/hexane mixture to afford 5-(3-tert-butyl-5-methoxycarbonyl-benzyl)-2,2-dimethyl-5-[3-[(triphenyl-methyl)thio]propyl]-[1,3]dioxane-4,6-dione X (14.0 g, 79%) as a white solid: ¹H NMR (CDCl₃) δ 0.7 (s, 3H), 1.3 (s, 9H), 1.2-1.3 (m, 2H), 1.5 (s, 3H), 2.0 (m, 2H), 2.2 (m, 2H), 3.3 (s, 2H), 3.8 (s, 3H), 7.2-7.4 (m, 16H), 7.6 (s, 1H), 7.8 (s, 1H); ¹³ NMR (CDCl₃) δ 24.8, 29.1, 29.4, 31.2, 31.4, 34.9, 40.3, 43.7, 52.3, 57.3, 66.8, 105.8, 126.0, 126.8, 128.0, 128.5, 129.6, 130.5, 132.3, 135.3, 144.8, 152.4, 167.1, 168.5.

2-(3-tert-Butyl-5-methoxycarbonyl-benzyl)-2-[3-[(triphenylmethyl)thio]propyl]-malonic acid XI

To a solution of 5-(3-tert-butyl-5-methoxycarbonyl-benzyl)-2,2-dimethyl-5-[3-[(triphenylmethyl)thio]propyl]-[1,3]dioxane-4,6-dione X (11 g, 16.5 mmol) in 1,4-dioxane (15 ml) was added a solution of sodium hydroxide (4.63 g, 115.5 mmol) in H₂O (15 mL) at 25° C., and the mixture was stirred at 100° C. for 1 hour. The solvent was removed under reduced pressure and the residue was partitioned between ethyl acetate and a 10% aqueous KHSO₄ solution. The organic layer was dried over MgSO₄, concentrated. The crude material was recrystallized from ethyl acetate/hexane mixture to afford 2-(3-tert-butyl-5-methoxycarbonyl-benzyl)-2-[3-[(triphenyl-methyl)thio]-propyl]malonic acid XI (9.0 g, 90%) as a white solid: ¹H NMR (CD₃OD) δ 1.4 (s, 9H), 1.4 (m, 2H), 1.6 (m, 2H), 2.1 (t, J=8.0 Hz, 2H), 3.2 (s, 2H), 7.1-7.4 (m, 16H), 7.7 (s, 1H), 7.9 (s, 1H); ¹³C NMR (CD₃OD) δ 24.8, 31.8 (3C), 32.4, 33.3, 35.6, 39.0, 59.5, 67.7, 126.2, 127.7, 128.9, 129.6, 130.7, 131.5, 132.9, 137.8, 146.2, 152.6, 170.1, 174.5.

2-(3-Tert-Butyl-5-methoxycarbonyl-benzyl)-5-[(triphenylmethyl)thio]pentanoic acid XII

A solution of 2-(3-tert-butyl-5-methoxycarbonyl-benzyl)-2-[3-[(triphenylmethyl)thio]propyl]-malonic acid XI (6.71 g, 11 mmol) in DMSO (10 ml) was stirred at 130° C. for 1.5 hours. The solvent was removed under reduced pressure and water was added to the residual oil. The precipitate was filtered off, washed with water, and dried under vacuum to afford 2-(3-tert-butyl-5-methoxycarbonyl-benzyl)-5-[(triphenylmethyl)thio]-pentanoic acid XII (5.86 g, 10.3 mmol, 94%) as a white solid: ¹H NMR (CD₃OD) δ 1.3 (s, 9H), 1.3-1.5 (m, 4H), 2.1 (m, 2H), 2.4 (m, 1H), 2.7 (m, 1H), 2.8 (m, 1H), 7.1-7.4 (m, 16H), 7.7 (s, 1H), 7.9 (s, 1H); ¹³C NMR (CD₃0D) δ 27.4, 31.7 (3C), 32.3, 32.7, 35.6, 39.2, 48.4, 67.7, 125.7, 127.7, 128.6, 128.9, 130.8, 131.6, 132.0, 140.8, 146.3, 152.7, 170.3, 178.8.

3-Carboxy-5-(1,1-dimethylethyl)-alpha-(3-mercaptopropyl)-benzenepropanoic acid

To a solution of 2-(3-tert-butyl-5-methoxycarbonyl-benzyl)-5-[(triphenylmethyl)thio]pentanoic acid XII (5.5 g, 9.7 mmol) in dichloromethane (30 mL) were added triisopropylsilane (2.4 mL, 11.6 mmol) and trifluoroacetic acid (10 mL), and the mixture was stirred at room temperature for 10 minutes. The solvent was removed under reduced pressure and the crude material was purified by silica gel chromatography (1% AcOH in Hexanes/EtOAc, 4:1) to afford 3-carboxy-5-(1,1-dimethylethyl)-alpha-(3-mercaptopropyl)-benzenepropanoic acid (1.7 g, 5.3 mmol, 55%) as a white solid: ¹H NMR (CD₃OD) δ 1.3 (s, 9H), 1.5-1.8 (m, 4H), 2.4 (m, 2H), 2.6-2.7 (m, 1H), 2.8-2.9 (m, 1H), 2.9-3.0 (m, 1H), 7.5 (s, 1H), 7.7 (s, 1H), 7.8 (s, 1H); ¹³C NMR (CD₃OD) δ 24.8, 31.7 (3C), 31.9, 32.9, 35.6, 39.5, 48.6, 125.7, 128.5, 131.6, 132.0, 140.9, 152.8, 170.3, 179.0. Elemental analysis calculated for C₁₇H₂₄O₄S: C, 62.93; H, 7.46; S, 9.88. Found: C, 63.02; H, 7.36; S, 9.82.

### Example 6

### In Vitro Inhibition of NAALADase Activity

Various compounds used in the inventive methods and pharmaceutical compositions have been tested for in vitro inhibition of NAALADase activity. The experimental protocol and some of the results are set forth in U.S. Pat. Nos. 5,672,592, 5,795,877, 5,863,536, 5,880,112, 5,902,817, 5,962,521, 6,025,344, 6,028,216 and 6,046,180, allowed U.S. patent applications Ser. Nos. 08/842,360, 09/002,147 and 09/050,009 for which the issue fees have been paid, and International Publications Nos. WO 97/48400, WO 99/33849 and WO 00/01668, the entire contents of which patents, patent applications and publications are herein incorporated by reference.

Other exemplary results are provided below in TABLE I.

TABLE I
IN VITRO INHIBITION OF NAALADASE ACTIVITY

| Compound | Kᵢ (nM) |
|---|---|
| 4-[4-(2,4-dicarboxybenzoyl)phenoxy]- | 1170 |
| 1,2-benzenedicarboxylic acid | |
| 2-[(4-carboxyphenyl)sulfonyl]-1,4- | 2370 |
| benzenedicarboxylic acid | |
| 2-[(2,5-dicarboxyphenyl)sulfonyl]-1,4- | 1870 |
| benzenedicarboxylic acid | |
| 4-[(2-carboxyphenyl)thio]-1,3- | 3980 |
| benzenedicarboxylic acid | |
| 2-[(2-carboxyphenyl)thio]-1,4- | 572 |
| benzenedicarboxylic acid | |
| 4-[3-[[3-(2,4-dicarboxyphenoxy)- | 3750 |
| propyl]-dithio]propoxy]-1,3- | |
| benzenedicarboxylic acid | |
| 5-(3-mercaptopropoxy)-1,3- | 3300 |
| benzenedicarboxylic acid | |
| 5-(2-mercaptoethoxy)-1,3- | 14500 |
| benzenedicarboxylic acid | |
| 5-[(hydroxyamino)-carbonyl]-1,3- | 1000 |
| benzenedicarboxylic acid | |
| 5-phosphono-1,3-benzenedicarboxylic | 14000 |
| acid | |
| 5-mercaptomethyl-1,3- | 6500 |
| benzenedicarboxylic acid | |
| 5-phosphonomethyl-1,3- | 3100 |
| benzenedicarboxylic acid | |
| 5-[(carboxymethyl)amino]-1,3- | 100000 |
| benzenedicarboxylic acid | |
| 5-[[(2-furanylmethyl)amino]methyl]- | 50000 |
| 1,3-benzenedicarboxylic acid | |
| 2-carboxymethyl-1,4- | 9000 |
| benzenedicarboxylic acid | |
| 5-[2-(hydroxyamino)-2-oxoethyl]-1,3- | 12000 |
| benzenedicarboxylic acid | |
| 4-(2-mercaptoethyl)-1,3- | 116 |
| benzenedicarboxylic acid | |
| 5-(2-mercaptoethyl)-1,3- | 5100 |
| benzenedicarboxylic acid | |

### Example 7

### Effect of NAALADase Inhibitors on Onset of Clinical Disease

The effect of NAALADase inhibitors on the onset of ALS was tested using the transgenic mice model of familial amyotrophic lateral sclerosis (FALS), which is detailed in Gurney, M., Annals of Neurology (1996) 39:147-157, and otherwise well known in the art. One month old transgenic SOD mice were treated with daily intraperitoneal injections of a vehicle (50 mM HEPES-buffered saline) or a NAALADase inhibitor (50 mg/kg 2-[[(2,3,4,5,6-pentafluorobenzyl)hydroxyphosphinyl]methyl]-pentanedioic acid (“Compound A” )). Clinical symptoms of the mice were monitored daily. The onset of clinical disease was scored by examining each mouse for its shaking of limbs when suspended in the air by its tail, cross spread of spinal reflexes, hindlimb paralysis, body weight and wheel running activity.

The results, set forth below in TABLE II, show that disease onset was delayed in mice treated with a NAALADase inhibitor.

TABLE II
EFFECT OF NAALADASE INHIBITOR ON ONSET OF CLINICAL
DISEASE

| | DISEASE | DISEASE | |
|---|---|---|---|
| | ONSET FOR | ONSET FOR | |
| | COMPOUND B | VEHICLE | |
| | TREATED MICE | TREATED MICE | |
| | (days of | (days of | |
| STUDY | age) | age) | DIFFERENCE |
| Study 1 | 221 | 189 | 32 |
| Study 2 | 166 | 141 | 25 |

### Example 8

### Effect of NAALADase Inhibitor on Survival and Clinical Symptoms

The effect of NAALADase inhibitors on survival and clinical symptoms was tested using again the transgenic mice model of FALS. one month old transgenic SOD mice were treated daily with a vehicle (50 mM HEPES-buffered saline) or a NAALADase inhibitor (30 mg/kg 2-(3-sulfanylpropyl)pentanedioic acid (“Compound B”)) p.o. Clinical symptoms of the mice were monitored twice a week. Such symptoms included shaking of limbs, gait, dragging of hind limbs, crossing of limbs, righting reflex and mortality. Gait and crossing of limbs were graded on an arbitrary scale ranging from 0 to 3, with 0 representing most normal and 3 representing least normal, e.g. severest difficulty in walking or crossing limbs. Righting reflex was measured by the time (seconds) it took the mice to right themselves when placed on their sides on a flat surface.

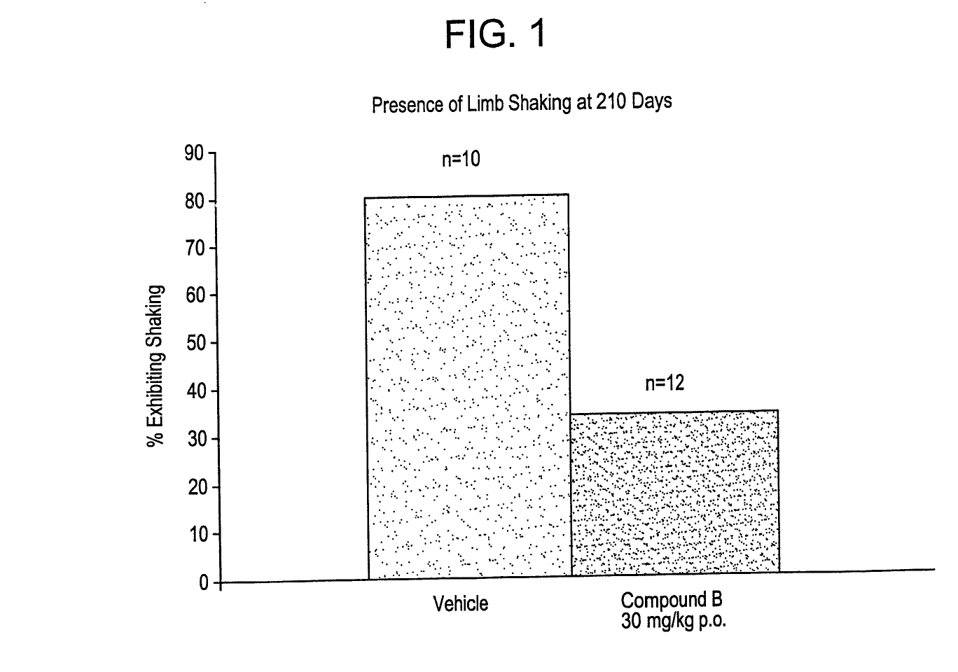

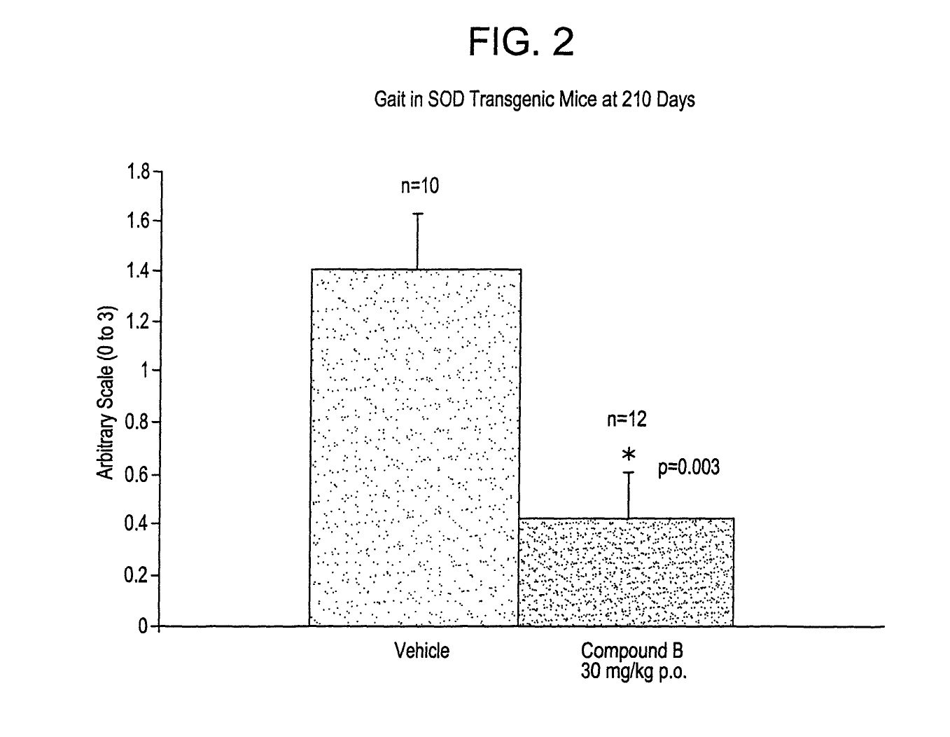

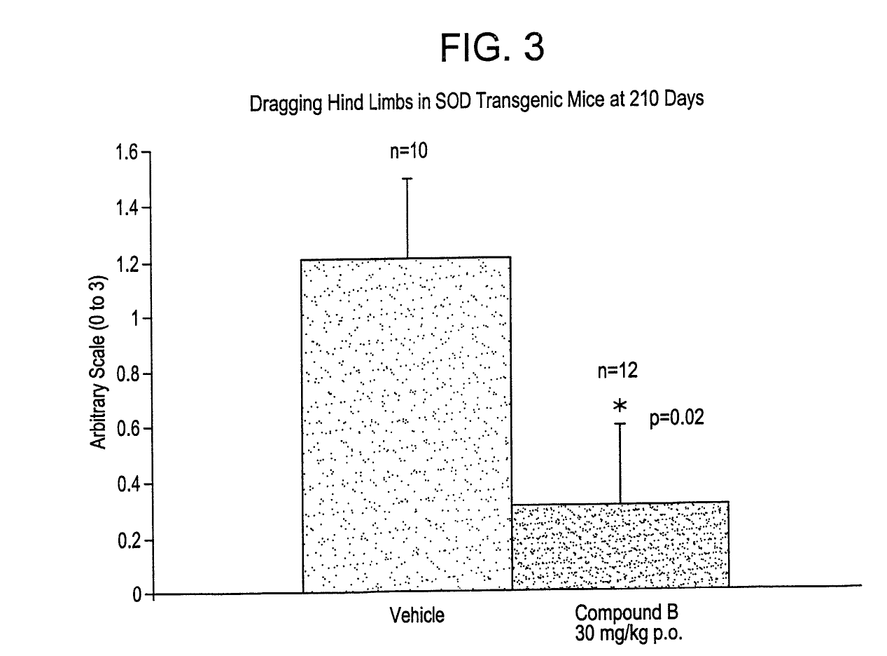

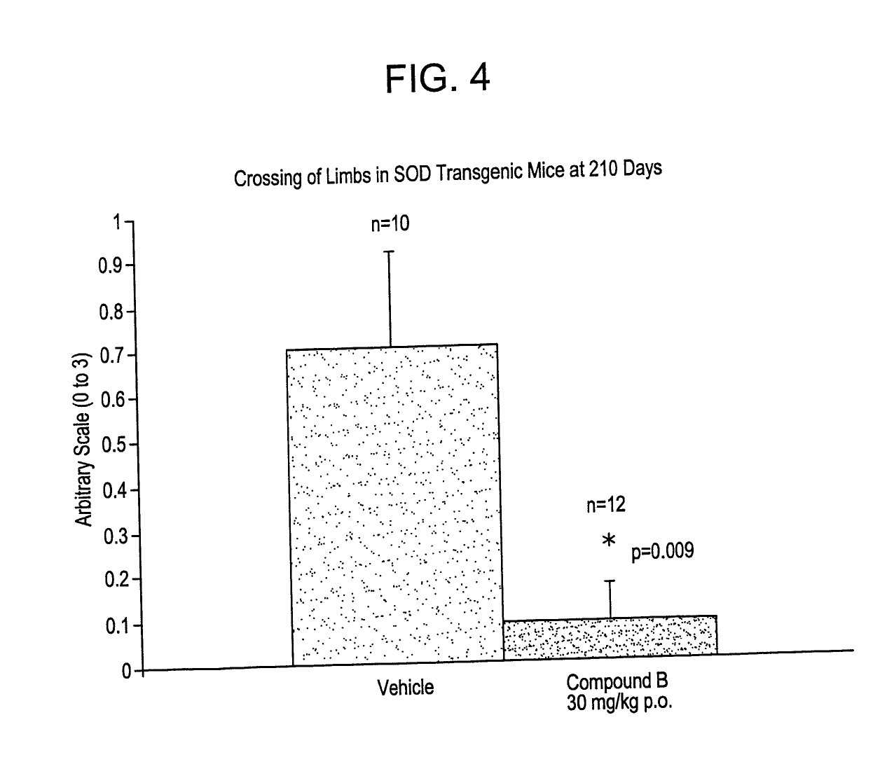

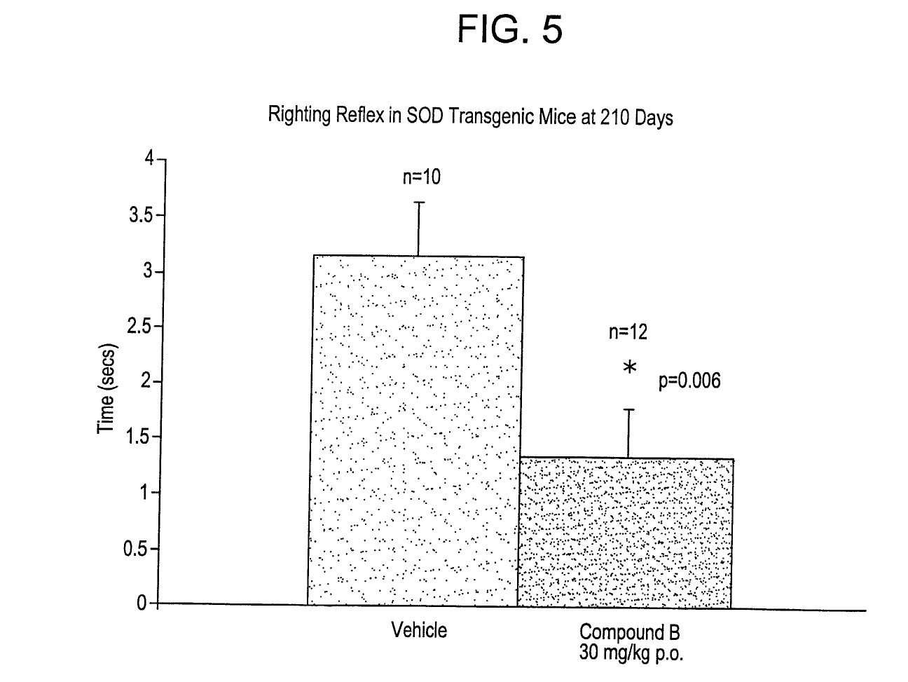

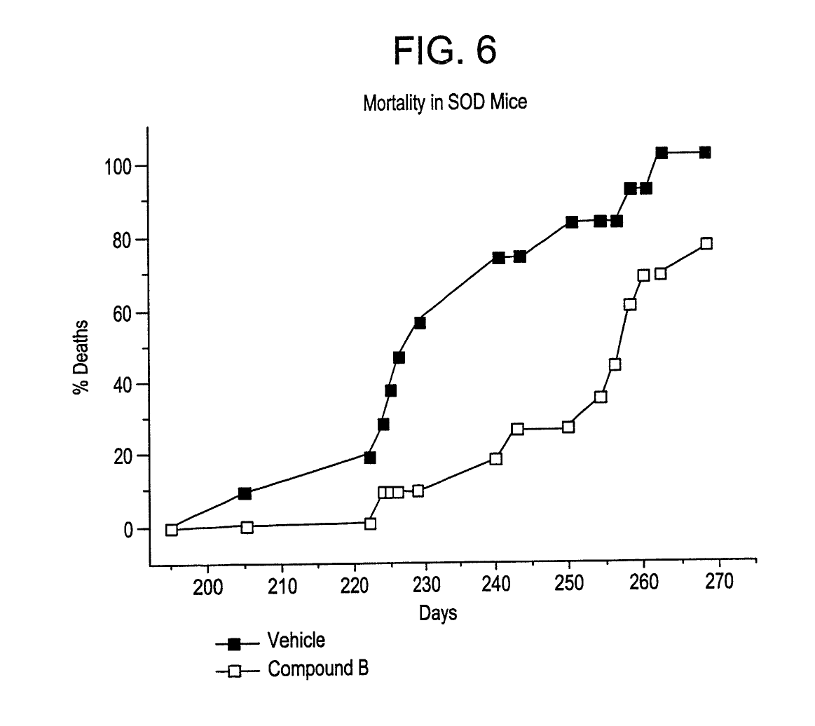

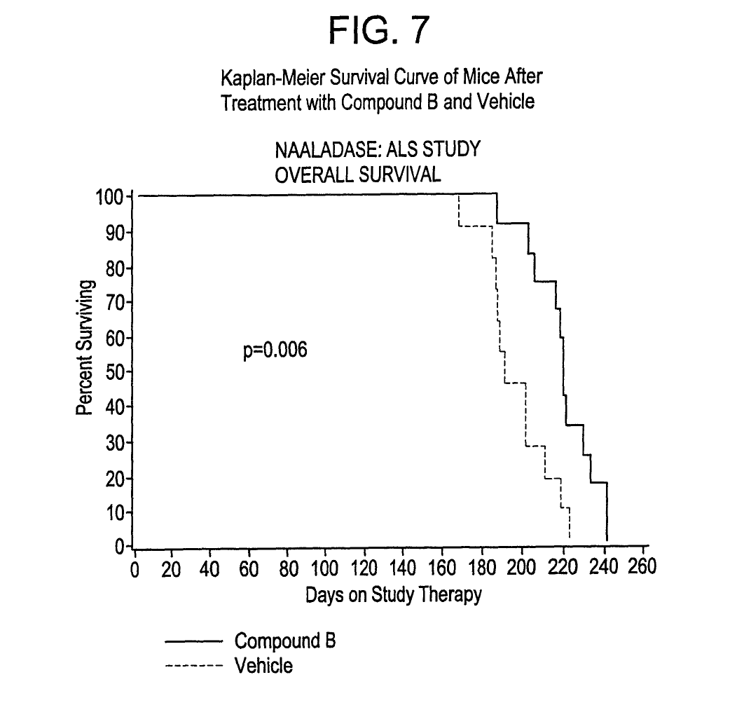

The results, set forth in FIGS. 1-7, show that survival was prolonged and clinical symptoms were delayed and attenuated in mice treated with a NAALADase inhibitor.

All publications, patents and patent applications identified above are herein incorporated by reference, as though set forth herein in full.

The invention being thus described, it will be apparent to those skilled in the art that the same may be varied in many ways without departing from the spirit and scope of the invention. Such variations are included within the scope of the following claims.

We claim:

1. A method for treating amyotrophic lateral sclerosis (ALS) comprising administering an effective amount of a NAALADase inhibitor to a mammal in need of such treatment.

2. The method of claim 1, wherein the NAALADase inhibitor is an acid containing a metal binding group.

3. The method of claim 1, wherein the NAALADase inhibitor is a compound of formula I 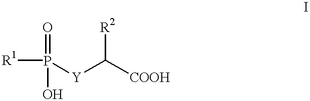
or an enantiomer or a pharmaceutically acceptable equivalent of said compound, wherein:
Y is CR³R⁴, NR⁵ or O;
R¹ is hydrogen, Cₗ-C₉ alkyl, C₂-C₉ alkenyl, C₃-C₈ cycloalkyl, C₅-C₇ cycloalkenyl, Ar, COOR⁶, NR⁶R⁷ or OR⁶, wherein said alkyl, alkenyl, cycloalkyl and cycloalkenyl are independently unsubstituted or substituted with one or more substituent (s), preferably, independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, COOR⁶, NR⁶R⁷ and Ar;
R² is hydrogen, C₁-C₆ alkyl, C₂-C₆ alkenyl, C₃-c₈ cycloalkyl, C₁-C₇ cycloalkenyl, Ar, halo or carboxy, wherein said alkyl, alkenyl, cycloalkyl and cycloalkenyl are independently unsubstituted or substituted with one or more substituent(s), preferably, independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, NR⁶R⁷ and Ar;
R³ and R⁴ are independently hydrogen or C₁-C₃ alkyl;
R⁵ is hydrogen or C₁-C₃ alkyl;
R⁶ and R⁷ are independently hydrogen, C₁-C₉ alkyl, C₂-C₉ alkenyl, C₃- cycloalkyl, C₅-C₇ cycloalkenyl or Ar, wherein said alkyl, alkenyl, cycloalkyl and cycloalkenyl are independently unsubstituted or substituted with one or more substituent(s), preferably, independently selected from the group consisting of carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy and Ar; and
Ar is selected from the group consisting of 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl and phenyl, wherein said Ar is unsubstituted or substituted with one or more substituent(s), preferably, independently selected from the group consisting of halo, hydroxy, nitro, trifluoromethyl, C₁-C₆ alkyl, C₂-C₆ alkenyl, Cₗ-C₆ alkoxy, C₂-C₆ alkenyloxy, phenoxy, benzyloxy, carboxy and N⁶R⁷.

4. The method of claim 3, wherein Y is CH₂.

5. The method of claim 4, wherein R² is —(CH₂)₂COOH.

6. The method of claim 5, wherein R¹ is hydrogen, C₁-C₄ alkyl, C₂-C₄ alkenyl, C₃-C₈ cycloalkyl, C₅-C₇ cycloalkenyl, benzyl, phenyl or OR⁶, wherein said alkyl, alkenyl, cycloalkyl, cycloalkenyl, benzyl and phenyl are independently unsubstituted or substituted with one or more substituent(s) independently selected from the group consisting of carboxy, C₃-C₈ cycloalkyl, C₁-C₇ cycloalkenyl, halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, NR⁶R⁷, benzyl and phenyl.

7. The method of claim 6, wherein the compound of formula I is selected from the group consisting of:
2-(phosphonomethyl)pentanedioic acid;
2-[[(2-carboxyethyl)hydroxyphosphinyl]methyl]-pentanedioic acid;
2-[(benzylhydroxyphosphinyl)methyl]pentanedioic acid;
2-[(phenylhydroxyphosphinyl)methyl]pentanedioic acid;
2-[[((hydroxy)phenylmethyl)hydroxyphosphinyl]-methyl]pentanedioic acid;
2-[(butylhydroxyphosphinyl)methyl]pentanedioic acid;
2-[[(3-methylbenzyl)hydroxyphosphinyl]methyl]-pentanedioic acid;
2-[(3-phenylpropylhydroxyphosphinyl)methyl]-pentanedioic acid;
2-[[(4-fluorophenyl)hydroxyphosphinyl]methyl]-pentanedioic acid;
2-[(methylhydroxyphosphinyl)methyl]pentanedioic acid;
2-[(phenylethylhydroxyphosphinyl)methyl]pentanedioic acid;
2-[[(4-methylbenzyl)hydroxyphosphinyl]methyl]-pentanedioic acid;
2-[[(4-fluorobenzyl)hydroxyphosphinyl]methyl]-pentanedioic acid;
2-[[(4-methoxybenzyl)hydroxyphosphinyl]methyl]-pentanedioic acid;
2-[[(3-trifluoromethylbenzyl)hydroxyphosphinyl]-methyl]pentanedioic acid;
2-[[4-trifluoromethylbenzyl)hydroxyphosphinyl]-methyl]pentanedioic acid;
2-[[(2-fluorobenzyl)hydroxyphosphinyl]methyl]-pentanedioic acid;
2-[[(2,3,4,5,6-pentafluorobenzyl)hydroxy-phosphinyl]methyl]pentanedioic acid; and
enantiomers and pharmaceutically acceptable equivalents.

8. The method of claim 1, wherein the NAALADase inhibitor is a compound of formula II 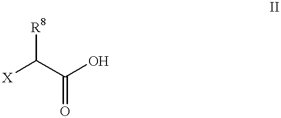
or an enantiomer or a pharmaceutically acceptable equivalent of said compound, wherein:
X is a moiety of formula III, IV or V 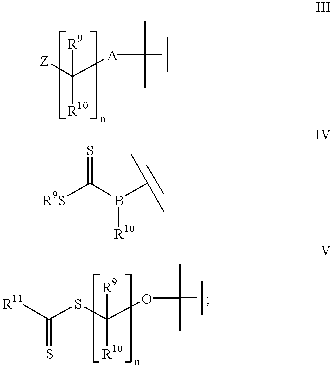
Z is SH, SO_{3H}, SO₂H, SOH, SO (NH) R¹² or S(NHR¹²)₂R¹³;
B is N or CR¹⁴;
A is O, S, CR¹⁵R¹⁶ or (CR₁₅R¹⁶)ₘS;
m and n are independently 0, 1, 2, 3 or 4;
R⁸, R⁹, R¹⁰, R¹¹, R¹², R¹⁴, R¹⁵ and R¹⁶ are independently hydrogen, Cₗ-C₉ alkyl, C₂-C₉ alkenyl, C₃-C₈ cycloalkyl, C₁-C₇ cycloalkenyl, Ar¹, hydroxy, carboxy, carbonyl, amino, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and
Ar¹ is a carbocyclic or heterocyclic moiety, which is unsubstituted or substituted with one or more substituent(s);
provided that when X is a moiety of formula III and A is 0, then n is 2, 3 or 4; when X is a moiety of formula III and A is S, then n is 2, 3 or 4; and when X is a moiety of formula III and A is (CR¹⁵R¹6)ₘS, then n is 0, 2, 3 or 4.

9. The method of claim 8, wherein:
X is a moiety of formula III;
n is 0, 1, 2 or 3;
Z is SH, SO₃H, SO₂H, SOH or S(NHR¹²)₂R¹³; and
A is O, S or CR¹⁵R¹⁶.

10. The method of claim 9, wherein Z is SH.

11. The method of claim 10, wherein R⁸ is —(CH₂)₂COOH.

12. The method of claim 10, wherein the compound of formula II is selected from the group consisting of:
2-(2-sulfanylethyl)pentanedioic acid;
3-(2-sulfanylethyl)-1,3,5-pentanetricarboxylic acid;
2-(2-sulfanylpropyl)pentanedioic acid;
2-(2-sulfanylbutyl)pentanedioic acid;
2-(2-sulfanyl-2-phenylethyl)pentanedioic acid;
2-(2-sulfanylhexyl)pentanedioic acid;
2-(2-sulfanyl-1-methylethyl)pentanedioic acid;
2-[1-(sulfanylmethyl)propyl]pentanedioic acid;
2-(3-sulfanylpentyl)pentanedioic acid;
2-(3-sulfanylpropyl)pentanedioic acid;
2-(3-sulfanyl-2-methylpropyl)pentanedioic acid;
2-(3-sulfanyl-2-phenylpropyl)pentanedioic acid;
2-(3-sulfanylbutyl)pentanedioic acid;
2-[3-sulfanyl-2-(phenylmethyl)propyl]pentanedioic acid;
2-[2-(sulfanylmethyl)butyl]pentanedioic acid;
2-[2-(sulfanylmethyl)pentyl]pentanedioic acid;
2-(3-sulfanyl-4-methylpentyl)pentanedioic acid; and
enantiomers and pharmaceutically acceptable equivalents.

13. The method of claim 1, wherein the NAALADase inhibitor is a compound of formula VI 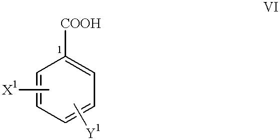
or an enantiomer or a pharmaceutically acceptable equivalent of said compound, wherein:
X¹ is —W—Z¹;
W is a bond or a linking group;
Z¹ is a terminal group; and
Y¹ is —COOH oriented meta or para relative to C-1.

14. The method of claim 13, wherein:
X¹ is —(CR¹⁷R¹⁸)ₙNH(CR¹⁹R²⁰)ₘCOOH, —PO(OH)OR²², —(CR¹⁷R¹⁸)ₙP(O)(OH) R²², —NH—(CR¹⁹R²⁰)ₘ-heteroaryl, —NH(P(O)(R²³)OH), —(CR¹⁷R¹⁸)ₙNH (P(O)(OH) R²³), —CON(R²²)(OH)—(CR¹⁷CR¹⁸)ₙCON(R²²)(OH), —(CR¹⁷R¹⁸)ₙSH or —O(CR¹⁹R²⁰)ₘSH, —SO₂NH-aryl, —N (C═O)—CH₂(C═O)-aryl, —SO₂NH-aryl, —N(C═O)—CH₂(C═O)-aryl, —O-aryl wherein aryl in —O-aryl is substituted by at least one of nitro, carboxy or 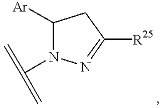
wherein X¹ is oriented meta or para relative to C-1;
m and n are independently 1-3, provided that when X¹ is —O(CR¹⁹R²⁰)mSH, then m is 2 or 3; R¹⁷, R¹⁸, R¹⁹, R²⁰, R²², R²³ and R²⁵ are independently hydrogen, C₁-C₆ alkyl, C₁-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituent(s); and
Y¹ is —COOH oriented meta or para relative to C-1.

15. The method of claim 13, wherein the compound of formula VI is selected from the group consisting of
2-[(4-carboxyphenyl)sulfonyl]-1,4-benzene-dicarboxylic acid;
2-[(2,5-dicarboxyphenyl)sulfonyl]-1,4-benzene-dicarboxylic acid;
1,2,4-benzenetricarboxylic acid;
2-[(2-carboxyphenyl)thio]-1,4-benzenedicarboxylic acid;
2-nitro-1,4-benzenedicarboxylic acid;
2-bromo-1,4-benzenedicarboxylic acid;
2-amino-1,4-benzenedicarboxylic acid;
2-sulfoterephthalic acid, monosodium salt;
2-carboxymethyl-1,4-benzenedicarboxylic acid;
2-[(2-furanylmethyl)-amino]-1,4-benzenedicarboxylic acid;
2-[(carboxymethyl)amino]-1,4-benzenedicarboxylic acid;
4-(4-nitrobenzoyl)-1,3-benzenedicarboxylic acid;
4-[4-(2,4-dicarboxybenzoyl)phenoxy]-1,2-benzene-dicarboxylic acid;
4-[[(2,4,6-trimethylphenyl)amino]carbonyl]-1,3-benzenedicarboxylic acid;
4-nitro-1,3-benzenedicarboxylic acid;
4-[(1-naphthalenylamino)-carbonyl]-1,3-benzene-dicarboxylic acid;
1,2,4-benzenetricarboxylic acid;
4-[(2-carboxyphenyl)thio]-1,3-benzenedicarboxylic acid;
4-[3-[[3-(2,4-dicarboxyphenoxy)propyl]dithio]-propoxy]-1,3-benzenedicarboxylic acid;
4-hydroxy-1,3-benzenedicarboxylic acid;
4-[(2-furanylmethyl)amino]-1,3-benzenedicarboxylic acid;
4-(2-mercaptoethyl)-1,3-benzenedicarboxylic acid;
5-[4,5-dihydro-5-(4-hydroxyphenyl)-3-phenyl-1H-pyrazol-1-yl]-1,3-benzenedicarboxylic acid;
5-(4,5-dihydro-3-methyl-5-phenyl-1H-pyrazol-1-yl)-1,3-benzenedicarboxylic acid;
5-[[(4-chloro-3-nitrophenyl)amino]sulfonyl]-1,3-benzenedicarboxylic acid;
5-[[[4-chloro-3-[[3-(2-methoxyphenyl)-1,3-dioxopropyl]amino]phenyl]amino]sulfonyl-1,3-benzenedicarboxylic acid;
5-[[3-[4-(acetylamino)phenyl]-1,3-dioxopropyl]amino]-1,3-benzenedicarboxylic acid;
5-acetylamino-1,3-benzenedicarboxylic acid; 5-[[(1-hydroxy-2-naphthalenyl)carbonyl]-methylamino]-1,3-benzenedicarboxylic acid;
5-(4-carboxy-2-nitrophenoxy)-1,3-benzenedicarboxylic acid;
5-sulfo-1,3-benzenedicarboxylic acid;
5-nitro-1,3-benzenedicarboxylic acid;
5-amino-1,3-benzenedicarboxylic acid;
1,3,5-benzenetricarboxylic acid;
5-[[(3-amino-4-chlorophenyl)amino]sulfonyl]-1,3-benzenedicarboxylic acid;
5-(3-mercaptopropoxy)-1,3-benzenedicarboxylic acid;
5-hydroxy-1,3-benzenedicarboxylic acid;
5-(2-mercaptoethoxy)-1,3-benzenedicarboxylic acid;
5-[(hydroxyamino)carbonyl]-1,3-benzenedicarboxylic acid;
5-phosphono-1,3-benzenedicarboxylic acid;
5-mercaptomethyl-1,3-benzenedicarboxylic acid;
5-phosphonomethyl-1,3-benzenedicarboxylic acid;
5-[[(carboxymethyl)amino]-methyl]-1,3-benzene-dicarboxylic acid;
5-[(carboxymethyl)amino]-1,3-benzenedicarboxylic acid;
5-[[(2-furanylmethyl)amino]-methyl]-1,3-benzene-dicarboxylic acid;
5-[2-(hydroxyamino)-2-oxoethyl]-1,3-benzene-dicarboxylic acid;
5-(2-mercaptoethyl)-1,3-benzenedicarboxylic acid; and
enantiomers and pharmaceutically acceptable equivalents.

16. The method of claim 1, wherein treating ALS is delaying onset of ALS or ALS symptom(s).

17. The method of claim 1, wherein treating ALS is slowing progression of ALS or ALS symptom(s).

18. The method of claim 1, wherein treating ALS is prolonging survival of an animal suffering from ALS.

19. The method of claim 1, wherein treating ALS is attenuating one or more ALS symptom(s).

20. A pharmaceutical composition comprising:
(i) an effective amount of a NAALADase inhibitor for treating amyotrophic lateral sclerosis (ALS); and
(ii) a pharmaceutically acceptable carrier.
